(19) United States
(12) Patent Application Publication
(10) Pub. No.: US 2020/0368198 A1
(43) Pub. Date: Nov. 26, 2020

(54) METHODS FOR INCREASING FERTILITY

(71) Applicant: NEWSOUTH INNOVATIONS PTY LIMITED, New South Wales (AU)

(72) Inventors: Lindsay Edward Wu, New South Wales (AU); David Andrew Sinclair, Chestnut Hill, MA (US); Hayden A. Homer, Queensland (AU)

(21) Appl. No.: 16/635,454

(22) PCT Filed: Jul. 31, 2018
(86) PCT No.: PCT/AU2018/050798

§ 371 Date: Jan. 30, 2020

(30) Foreign Application Priority Data
Jul. 31, 2017 (AU) 2017903013

(51) Int. Cl. A61K 31/353; C12N 5/075

(52) U.S. Cl. CPC A61K 31/353; A61K 45/06; C12N 5/0609

(57) ABSTRACT

The present invention relates to a method of increasing fertility, or reducing rate of decline in fertility, or restoring fertility, of a female subject, comprising administering to the subject an effective amount of an agent which elevates SIRT2 activity or SIRT2 expression, and to compositions and kits for increasing fertility, or reducing rate of decline in fertility, or restoring fertility.

The present application claims priority from Australian provisional application no. 2017903013, the entirety of which is incorporated herein by reference.

### FIELD

The present invention relates to a method of increasing fertility of a female subject, to a method of increasing oocyte quality in a female subject, and to a composition for increasing fertility of a female subject.

### BACKGROUND

The strongest determinant of female reproductive success is age, with an acute decline in fertility beyond the middle of the third decade of life in humans. With a constant trend towards an increased age of maternity across the world, female infertility is a growing problem, resulting in a growing demand for assisted reproductive technologies (ART) such as in vitro fertilisation (IVF). The success of IVF is drastically limited by an age-dependent decline in oocyte quality, with a 26% success rate for women aged under 30 compared to a less than 1% success rate for women aged over 45. This decline in success is primarily driven by issues of oocyte quality, as the age-dependent decline in IVF success is restored when donor oocytes from younger women are used.

The molecular cause of this decline in oocyte quality with advancing age is not clear, with factors thought to be involved in this decline including an increase in reactive oxygen species (ROS), declining mitochondrial bioenergetics, and an impaired ability to accurately segregate chromosomes during meiosis. This latter hypothesis is evidenced by an increased rate of aneuploidy in oocytes, and the increased incidence of offspring born with chromosomal abnormalities such as Trisomy 21, which causes Down's Syndrome, with advanced age.

In veterinary practice and in agriculture, female fertility is rate limiting in the breeding of animals with favourable characteristics, for example, thoroughbred horses. Certain breeds of animals also have impaired fertility, for example dairy producing cattle breeds. Fertility issues may also limit the production of animals for meat production, for example pigs, or dairy production, for example dairy cows. Improving female fertility would therefore be beneficial to agricultural production, veterinary practice, and the breeding of racing and companion animals.

What is needed are methods for increasing fertility of a female subject, or reducing the rate of decline in fertility of a female subject with age. It would also be advantageous to provide a method for increasing oocyte quality.

### SUMMARY

The inventors have found that increasing Sirtuin 2 (SIRT2) activity or expression in female subjects results in an increase in oocyte yield from the subject, an increase in the quality of oocytes produced by the subject, and an increase in the fertility of the subject.

Accordingly, a first aspect of the present invention provides a method of increasing fertility, reducing rate of decline in fertility, or restoring fertility, of a female subject, the method comprising administering to the subject an effective amount of an agent which elevates SIRT2 activity or SIRT2 expression in the subject.

An alternative first aspect provides an agent which elevates SIRT2 activity or SIRT2 expression in a female subject for use in increasing fertility, reducing rate of decline in fertility, or restoring fertility, of a female subject, or use of an agent which elevates SIRT2 activity or SIRT2 expression in a female subject in the manufacture of a medicament for increasing fertility, reducing rate of decline in fertility, or restoring fertility, of a female subject.

A second aspect of the present invention provides a method of increasing oocyte yield and/or oocyte quality, or reducing rate of decline in oocyte yield and/or oocyte quality, in a female subject, the method comprising administering to the subject an effective amount of an agent which elevates SIRT2 activity or SIRT2 expression in the subject.

An alternative second aspect provides an agent which elevates SIRT2 activity or SIRT2 expression in a female subject for use in increasing oocyte yield and/or oocyte quality, or reducing rate of decline in oocyte yield and/or oocyte quality, in a female subject, or use of an agent which elevates SIRT2 activity or SIRT2 expression in a female subject in the manufacture of a medicament for increasing oocyte yield and/or oocyte quality, or reducing rate of decline in oocyte yield and/or oocyte quality, in a female subject.

A third aspect provides a method of preventing or reducing the occurrence of aneuploidy in an oocyte of a female subject, the method comprising administering to the subject an effective amount of an agent which elevates SIRT2 activity or SIRT2 expression in the subject.

An alternative third aspect provides an agent which elevates SIRT2 activity or SIRT2 expression in a female subject for use in preventing or reducing the occurrence of aneuploidy in oocytes of a female subject, or use of an agent which elevates SIRT2 activity or SIRT2 expression in a female subject in the manufacture of a medicament for preventing or reducing the occurrence of aneuploidy in oocytes of a female subject.

A fourth aspect of the present invention provides a method of treating or preventing infertility in a female subject suffering from infertility or a decline in fertility, or at risk of suffering from infertility or a decline in fertility, comprising administering to the subject an effective amount of an agent which elevates SIRT2 activity or SIRT2 expression the subject.

An alternative fourth aspect provides an agent which elevates SIRT2 activity or SIRT2 expression in a female subject for use in treating or preventing infertility in a female subject suffering from a loss of fertility or a decline in fertility, or use of an agent which elevates SIRT2 activity or SIRT2 expression in a female subject in the manufacture of a medicament for treating or preventing infertility in a female subject suffering from a loss of fertility or a decline in fertility.

A fifth aspect provides a method of reducing rate of decline in BubR1 activity in oocytes of a female subject suffering from a decline in fertility, or at risk of suffering from a decline in fertility, comprising administering to the subject an effective amount of an agent which elevates SIRT2 activity or SIRT2 expression in the subject.

An alternative fifth aspect provides an agent which elevates SIRT2 activity or SIRT2 expression in a female subject for use in reducing rate of decline in BubR1 activity in oocytes of a female subject suffering from a decline in fertility, or at risk of suffering from a decline in fertility, or use of an agent which elevates SIRT2 activity or SIRT2 expression in a female subject in the manufacture of a medicament for reducing rate of decline in BubR1 activity in oocytes of a female subject suffering from a decline in fertility, or at risk of suffering from a decline in fertility.

A sixth aspect provides a method of promoting regeneration, de novo generation or development of ovarian follicles in a female subject, comprising administering to the subject an effective amount of an agent which elevates SIRT2 activity or SIRT2 expression in the subject.

An alternative sixth aspect provides an agent which elevates SIRT2 activity or SIRT2 expression in a female subject for use in promoting regeneration, de novo generation or development of ovarian follicles in a female subject, or use of an agent which elevates SIRT2 activity or SIRT2 expression in a female subject in the manufacture of a medicament for promoting regeneration, de novo generation or development of ovarian follicles in a female subject.

A seventh aspect provides a method of increasing pregnancy success rate of a female subject(e.g. in a female subject suffering from a decline in fertility), comprising administering to the subject an effective amount of an agent which elevates SIRT2 activity or SIRT2 expression in the subject.

An alternative seventh aspect provides an agent which elevates SIRT2 activity or SIRT2 expression in a female subject for use in increasing pregnancy success rate of a female subject, or use of an agent which elevates SIRT2 activity or SIRT2 expression in a female subject in the manufacture of a medicament for increasing pregnancy success rate of a female subject.

An eighth aspect provides a method of increasing BubR1 activity in an oocyte, comprising introducing into the oocyte an effective amount of an agent which elevates SIRT2 activity or SIRT2 expression in the oocyte.

A ninth aspect provides a method of increasing the fertilisation potential of an oocyte, the method comprising introducing into the oocyte an effective amount of an agent which elevates SIRT2 activity or SIRT2 expression in the oocyte.

A tenth aspect provides a method of fertilizing an oocyte in vitro, comprising introducing into the oocyte a donor sperm and an effective amount of an agent which elevates SIRT2 activity or SIRT2 expression in the oocyte.

An eleventh aspect provides a method of increasing the probability that a zygote produced by fertilization of an oocyte in vitro will progress to a full term pregnancy following implantation, comprising introducing into the oocyte prior to, during, or after, fertilisation of the oocyte, an effective amount of an agent which elevates SIRT2 activity or SIRT2 expression in the oocyte.

A twelfth aspect provides a method of increasing fertility, reducing rate of decline in fertility, or restoring fertility, of a female subject, the method comprising administering to the subject an effective amount of an NAD⁺ agonist.

An alternative twelfth aspect provides an NAD+ agonist for use in increasing fertility, reducing rate of decline in fertility, or restoring fertility, of a female subject, or use of an NAD⁺ agonist in the manufacture of a medicament for increasing fertility, reducing rate of decline in fertility, or restoring fertility, of a female subject.

A thirteenth aspect of the present invention provides a method of increasing oocyte yield and/or oocyte quality, or reducing rate of decline in oocyte yield and/or oocyte quality, in a female subject, the method comprising administering to the subject an effective amount of an NAD⁺ agonist.

An alternative thirteenth aspect provides an NAD⁺ agonist for use in increasing oocyte yield and/or oocyte quality, or reducing rate of decline in oocyte yield and/or oocyte quality, in a female subject, or use of an NAD⁺ agonist in the manufacture of a medicament for increasing oocyte yield and/or oocyte quality, or reducing rate of decline in oocyte yield and/or oocyte quality, in a female subject.

A fourteenth aspect provides a method of preventing or reducing the occurrence of aneuploidy in an oocyte of a female subject, the method comprising administering to the subject an effective amount of an NAD⁺ agonist.

An alternative fourteenth aspect provides an NAD⁺ agonist for use in preventing or reducing the occurrence of aneuploidy in oocytes of a female subject, or use of an NAD⁺ agonist in the manufacture of a medicament for preventing or reducing the occurrence of aneuploidy in oocytes of a female subject.

A fifteenth aspect of the present invention provides a method of treating or preventing infertility in a female subject suffering from a loss of fertility or a decline in fertility, or at risk of suffering from a loss of fertility or a decline in fertility, comprising administering to the subject an effective amount of an NAD⁺ agonist.

An alternative fifteenth aspect provides an NAD⁺ agonist for use in treating or preventing infertility in a female subject suffering from a loss of fertility or a decline in fertility, or use of an NAD⁺ agonist in the manufacture of a medicament for treating or preventing infertility in a female subject suffering from a loss of fertility or a decline in fertility.

A sixteenth aspect provides a method of reducing rate of decline in BubR1 activity in oocytes of a female subject suffering from a decline in fertility, or at risk of suffering from a decline in fertility, comprising administering to the subject an effective amount of an NAD⁺ agonist.

An alternative sixteenth aspect provides an NAD⁺ agonist for use in reducing rate of decline in BubR1 activity in oocytes of a female subject suffering from a decline in fertility, or at risk of suffering from a decline in fertility, or use of an NAD⁺ agonist in the manufacture of a medicament for reducing rate of decline in BubR1 activity in oocytes of a female subject suffering from a decline in fertility, or at risk of suffering from a decline in fertility.

A seventeenth aspect provides a method of promoting regeneration, de novo generation or development of ovarian follicles in an adult female subject, comprising administering to the subject an effective amount of an NAD⁺ agonist.

An alternative seventeenth aspect provides NAD⁺ agonist for use in promoting regeneration, de novo generation or development of ovarian follicles in a female subject, or use of an NAD⁺ agonist in the manufacture of a medicament for promoting regeneration, de novo generation or development of ovarian follicles in a female subject.

An eighteenth aspect provides a method of increasing pregnancy success rate of a female subject (e.g. a female subject suffering from a decline in fertility), comprising administering to the subject an effective amount of an NAD⁺ agonist.

An alternative eighteenth aspect provides an NAD⁺ agonist for use in increasing pregnancy success rate of a female subject, or use of an NAD⁺ agonist in the manufacture of a medicament for increasing pregnancy success rate of a female subject.

A nineteenth aspect provides a method of increasing BubR1 activity in an oocyte, comprising introducing into the oocyte an effective amount of an NAD⁺ agonist.

A twentieth aspect provides a method of increasing the fertilisation potential of an oocyte, the method comprising introducing into the oocyte an effective amount of an NAD⁺ agonist.

A twenty first aspect provides a method of fertilizing an oocyte in vitro, comprising introducing into the oocyte a donor sperm and an effective amount of an NAD⁺ agonist.

A twenty second aspect provides a method of increasing the probability that a zygote produced by fertilization of an oocyte in vitro will progress to a full term pregnancy following implantation, comprising introducing into the oocyte prior to, during, or after, fertilisation of the oocyte, an effective amount of an NAD⁺ agonist.

A twenty third aspect provides a method of increasing fertility, reducing rate of decline in fertility, or restoring fertility, of a female subject, the method comprising administering to the subject an effective amount of an NAD⁺ precursor.

An alternative twenty third aspect provides an NAD+ precursor for use in increasing fertility, reducing rate of decline in fertility, or restoring fertility, of a female subject, or use of an NAD⁺ precursor in the manufacture of a medicament for increasing fertility, reducing rate of decline in fertility, or restoring fertility, of a female subject.

A twenty fourth aspect of the present invention provides a method of increasing oocyte yield and/or oocyte quality, or reducing rate of decline in oocyte yield and/or oocyte quality, in a female subject, the method comprising administering to the subject an effective amount of an NAD⁺ precursor.

An alternative twenty fourth aspect provides an NAD⁺ precursor for use in increasing oocyte yield and/or oocyte quality, or reducing rate of decline in oocyte yield and/or oocyte quality, in a female subject, or use of an NAD⁺ precursor in the manufacture of a medicament for increasing oocyte yield and/or oocyte quality, or reducing rate of decline in oocyte yield and/or oocyte quality, in a female subject.

A twenty fifth aspect provides a method of preventing or reducing the occurrence of aneuploidy in an oocyte of a female subject, the method comprising administering to the subject an effective amount of an NAD⁺ precursor.

An alternative twenty fifth aspect provides an NAD⁺ precursor for use in preventing or reducing the occurrence of aneuploidy in oocytes of a female subject, or use of an NAD⁺ precursor in the manufacture of a medicament for preventing or reducing the occurrence of aneuploidy in oocytes of a female subject.

A twenty sixth aspect of the present invention provides a method of treating or preventing infertility in a female subject suffering from a loss of fertility or a decline in fertility, or at risk of suffering from a loss of fertility or a decline in fertility, comprising administering to the subject an effective amount of an NAD⁺ precursor.

An alternative twenty sixth aspect provides an NAD⁺ precursor for use in treating or preventing infertility in a female subject suffering from a loss of fertility or a decline in fertility, or use of an NAD⁺ precursor in the manufacture of a medicament for treating or preventing infertility in a female subject suffering from a loss of fertility or a decline in fertility.

A twenty seventh aspect provides a method of reducing rate of decline in BubR1 activity in oocytes of a female subject suffering from a decline in fertility, or at risk of suffering from a decline in fertility, comprising administering to the subject an effective amount of an NAD⁺ precursor.

An alternative twenty seventh aspect provides an NAD⁺ precursor for use in reducing rate of decline in BubR1 activity in oocytes of a female subject suffering from a decline in fertility, or at risk of suffering from a decline in fertility, or use of an NAD⁺ precursor in the manufacture of a medicament for reducing rate of decline in BubR1 activity in oocytes of a female subject suffering from a decline in fertility, or at risk of suffering from a decline in fertility.

A twenty eighth aspect provides a method of promoting regeneration, de novo generation or development of ovarian follicles in an adult female subject, comprising administering to the subject an effective amount of an NAD⁺ precursor.

An alternative twenty eighth aspect provides NAD⁺ precursor for use in promoting regeneration, de novo generation or development of ovarian follicles in a female subject, or use of an NAD⁺ precursor in the manufacture of a medicament for promoting regeneration, de novo generation or development of ovarian follicles in a female subject.

An twenty ninth aspect provides a method of increasing pregnancy success rate of a female subject (e.g. a female subject suffering from a decline in fertility), comprising administering to the subject an effective amount of an NAD⁺ precursor.

An alternative twenty ninth aspect provides an NAD⁺ precursor for use in increasing pregnancy success rate of a female subject, or use of an NAD⁺ precursor in the manufacture of a medicament for increasing pregnancy success rate of a female subject.

A thirtieth aspect provides a method of increasing BubR1 activity in an oocyte, comprising introducing into the oocyte an effective amount of an NAD⁺ precursor.

A thirty first aspect provides a method of increasing the fertilisation potential of an oocyte, the method comprising introducing into the oocyte an effective amount of an NAD⁺ precursor.

A thirty second aspect provides a method of fertilizing an oocyte in vitro, comprising introducing into the oocyte a donor sperm and an effective amount of an NAD⁺ precursor.

A thirty third aspect provides a method of increasing the probability that a zygote produced by fertilization of an oocyte in vitro will progress to a full term pregnancy following implantation, comprising introducing into the oocyte prior to, during, or after, fertilisation of the oocyte, an effective amount of an NAD⁺ precursor.

A thirty fourth aspect provides a method of improving or enhancing the ability of an oocyte to form a blastocyst during in vitro fertilisation (IVF), comprising introducing into the oocyte an effective amount of an agent which elevates SIRT2 activity or SIRT2 expression in oocytes.

A thirty fifth aspect provides a method of improving or enhancing the ability of an oocyte to form a blastocyst during in vitro fertilisation (IVF), comprising introducing into the oocyte an effective amount of an NAD⁺ agonist.

A thirty sixth aspect provides a method of improving or enhancing the ability of an oocyte to form a blastocyst during in vitro fertilisation (IVF), comprising introducing into the oocyte an effective amount of an NAD⁺ precursor.

A thirty seventh aspect provides a composition for fertilization of an oocyte in vitro, comprising an agent which elevates SIRT2 activity or SIRT2 expression in oocytes.

A thirty eighth aspect provides a composition for fertilization of an oocyte in vitro, comprising an NAD+ agonist.

A thirty ninth aspect provides a composition for fertilization of an oocyte in vitro, comprising an NAD+ precursor.

A fortieth aspect provides a composition for increasing fertility of a female subject, comprising an agent which elevates SIRT2 activity or SIRT2 expression in oocytes.

A forty first aspect provides a composition for increasing fertility of a female subject, comprising an NAD+ agonist.

A forty second aspect provides a composition for increasing fertility of a female subject, comprising an NAD+ precursor.

A forty third aspect provide a kit for increasing fertility of a female subject, comprising an agent which elevates SIRT2 activity or SIRT2 expression in oocytes.

A forty fifth aspect provides a kit for increasing fertility of a female subject, comprising an NAD⁺ agonist.

A forty sixth aspect provides a kit for increasing fertility of a female subject, comprising an NAD⁺ precursor.

### BRIEF DESCRIPTION OF THE FIGURES

FIG. 1. A) is a Western blot of oocyte extracts showing that oocytes from SIRT2-Tg animals display elevated levels of BubR1 at 4 hr post germinal vesicle breakdown (GVBD). B) is a graph showing oocyte yield from 4 month old PMSG super-ovulated SIRT2-Tg females. C) is a graph showing polar body extrusion rates in oocytes from WT and SIRT2-Tg animals. D) is a graph and photograph showing DCFDA staining for reactive oxygen species (ROS) in oocytes from WT and SIRT2-Tg mice, which correlates with E), which is a graph showing elevated G6PD enzyme activity in oocytes. Error bars are SD.

FIG. 2. A) is a graph showing oocyte yield from 14 month-old PMSG stimulated WT and SIRT2-Tg littermate females. Oocytes were then assessed for meiotic progression through B) germinal vesicle breakdown (GVBD) rates and C) polar body extrusion (PBE) rates. D) is a graph showing MI oocyte staining for tubulin, kinetochores and DNA, and assessment for rates of abnormal spindle assembly. E) is an image showing MI oocyte staining for tubulin, kinetochores and DNA from PMSG stimulated SIRT1-Tg and WT littermate females. F) is a graph showing aneuploidy rates in oocytes from 14 month-old SIRT2-Tg and WT animals. G) is a graph showing cumulative pregnancy rates in female SIRT2-Tg and WT animals during repeated mating rounds, assessed from 15 months of age. H) is an image of stained ovaries from wild-type and SIRT2-Tg mice. Error bars are SD.

FIG. 3. A) is a graph showing oocyte yield in 14 month-old mice over-expressing the nuclear localised NAD⁺ biosynthetic enzyme NMNAT1. B) is a graph showing oocyte yield in 14 month-old mice over-expressing the mitochondrial localised NAD⁺ biosynthetic enzyme NMNAT3.

C) and D) are graphs showing oocyte yield in C57BL6 and SwissTacAusB mice, respectively, following treatment with or without NMN. Aged, 15 month old WT animals were treated with the NAD⁺ precursor nicotinamide mononucleotide (NMN) through addition to drinking water (2 g/L, 4 weeks), and stimulated with PMSG to determine oocyte yield in both the C) C57BL6 strain and D) SwissTacAusB strain of mice.

E) is a graph showing oocyte yield in high fat fed SwissTacAusB mice. 3 month-old SwissTacAusB females were maintained on chow diet or subjected to 3 months of high fat feeding in the presence or absence of NMN (drinking water, 2 g/L), and oocyte yield assessed following PMSG stimulation. F) is an image showing MII Oocytes from untreated or NMN treated (2 g/L drinking water, 4 weeks) 16 month-old C57BL6 females stained for tubulin (green), kinetochores (red) or DNA (blue) to assess abnormal spindle assembly. Error bars are SD.

FIG. 4. A) is a graph showing litter size. C57BL6 mice were maintained on normal drinking water supplemented with or without NMN (2 g/L) from 2 months of age. From 4 months of age, animals were timed mated with proven stud male C57BL6. Pregnancy was confirmed using micro-ultrasound for the presence of a foetal heartbeat, and the number of pups born in subsequent litters recorded. B) is a graph showing body weights of pups from breeding trials at 12 days of age.

FIG. 5. Offspring from NMN treated females, or non-NMN treated females, were maintained on a chow diet or subjected to feeding of a high fat diet (HFD) from 8 weeks of age. A) is a graph of body weights of animals until 23 weeks of age. B) is a graph of fat mass of animals after 7 weeks of HFD feeding. C) is a graph of glucose tolerance test in animals at 7 weeks after chow or HFD feeding (2 g/kg, 6 hr fast). D) is a graph of area under the curve for glucose tolerance tests. Each group represents offspring from at least 7 different females. Error bars are SD.

FIG. 6 is a graph showing the numbers of cumulus oocyte complexes released from mice treated with or without doxorubicin, in the presence or absence of NMN. Data were analysed by 2-way ANOVA with a post-hoc Tukey test.

FIG. 7 is a graph showing proportions (numbers showing % of total) of harvested oocytes achieving germinal vesicle breakdown at indicated timepoints, following release from IBMX.

FIG. 8 is a graph showing proportions of oocytes achieving polar body extrusion at indicated timepoints, following GVBD (see FIG. 7).

FIG. 9 is a graph showing the number of primordial follicles counted in ovarian H&E sections of animals treated as indicated.

FIG. 10 is a graph showing the number of follicles counted in H&E stained ovarian sections at each indicated stage of development, from mice treated as indicated.

FIG. 11 is a graph showing the number of live pups born per litter to female C57BL6 mice which were treated with doxorubicin and/or NMN as indicated, and mated.

FIG. 12 is a flow diagram of the design for mating trial experiments to address whether changes in oocyte quality and function translate into differences in fertility, and the ability to achieve pregnancy.

FIG. 13 is a graph showing the number of mating rounds required to achieve pregnancy for control mice, and mice treated with doxorubicin and/or NMN as indicated.

FIG. 14 is a graph showing body weights of pups at day 12 of age, following birth to females treated with doxorubicin and/or NMN as indicated. Day 12 body weights are an indicator of offspring health.

FIG. 15 is a graph showing numbers of cumulus oocyte complexes released from mice treated with or without cisplatin, in the presence or absence of NMN. Data were analysed by 2-way ANOVA with a post-hoc Tukey test.

FIG. 16 is a graph showing proportions (numbers showing % of total) of harvested oocytes achieving germinal vesicle breakdown at indicated time-points, following release from IBMX.

FIG. 17 is a graph showing proportions (numbers showing % of total) of harvested oocytes achieving polar body extrusion at indicated time-points, following completion of GVBD.

FIG. 18 is a graph showing oocyte yield following doxorubicin treatment (10 mg/kg, i.p.) in wild-type mice, or mice genetically engineered to over-express the nuclear NAD+ biosynthetic enzyme NMNAT1.

FIG. 19 is a graph showing oocyte yield following doxorubicin treatment (10 mg/kg, i.p.) in wild-type mice, or mice genetically engineered to over-express the mitochondrial NAD+ biosynthetic enzyme NMNAT3.

FIG. 20 is a schematic diagram showing the experimental design to test reversal of infertility. Eight week-old C57BL6 mice received chemotherapy or vehicle, and four weeks later, NMN treatment for an additional four weeks.

FIG. 21 is a graph showing primordial follicle numbers in ovarian histology sections taken from mice treated with doxorubicin alone, followed by NMN four weeks later.

FIG. 22 is a graph showing oocyte yield in mice treated treated with cisplatin alone (5 mg/kg, i.p.), followed by NMN 4 weeks later, and oocyte yield assessed a further 2 months later. **p<0.01, 2 way ANOVA with Tukey test.

FIG. 23 is a graph showing the number of pups born per female mouse treated as in FIG. 22 following 6 mating rounds with a male stud of proven fertility. *p<0.05, 2 way ANOVA with Tukey test.

FIG. 24 is a graph showing the number of pups born per litter in mice treated with or without cyclophosphamide (75 mg/kg, i.p. injection) at seven weeks of age, followed four weeks later by treatment with the NAD+ raising compound NMN for two months. These data indicate the ability of NAD+ raising compounds to reverse, rather than just prevent, infertility caused by chemotherapy treatment.

FIG. 25 is an image of a Western blot for BubR1 in 4 hr post-GVBD oocytes from control (WT) or SIRT2-Tg mice.

FIG. 26 is a graph showing oocyte (COC) yield in ovaries from 14 month-old WT control or SIRT2-Tg mice.

FIG. 27 is a graph showing meiosis I progression rates, as determined by proportion of oocytes achieving germinal vesicle breakdown, in COCs from 14 month old WT control or SIRT2-Tg mice. Numbers given are % of total oocytes.

FIG. 28 is a graph showing meiosis II progression rates, as determined by proportion of oocytes achieving polar body extrusion, in COCs from 14 month old WT control or SIRT2-Tg mice. Numbers given are % of total oocytes.

FIG. 29 is images showing spindle formation in oocytes from aged control (WT) or SIRT2-Tg littermates. Spindles are highlighted in green, using immunostaining for β-tubulin, kinetochores are in red, and chromosomes are in blue (Hoescht stain). Images are confocal sections through oocytes.

FIG. 30 is an image and graph showing aneuploidy rates in oocytes from aged (15 month old) control (WT) or SIRT2-Tg littermates. Aneuploidy was assessed through manual counting of chromosome pairs in monastrol treated oocytes. Numbers given are % of oocytes with either euploid (normal) or aneuploid (abnormal) chromosome numbers.

FIG. 31 is an image showing DCFDA staining for reactive oxygen species in oocytes from control (WT) or SIRT2-Tg littermates, following H₂O₂ treatment.

FIG. 32 is a graph showing Glucose 6 phosphate dehydrogenase (G6PD) enzymatic activity in oocytes from control (WT) and SIRT2-Tg oocytes. G6PD carries out detoxification of reactive oxygen species, and generates metabolic precursors for nucleotide biosynthesis.

FIG. 33 is a graph showing pregnancy success rates in aged (16 month old) SIRT2-Tg and WT littermate controls, over 5 mating rounds.

FIG. 34 is a schematic diagram of the study design for treatment of aged mice with NMN. 15 month old C57BL6 female mice were treated with NMN at 15 months of age for 3 weeks, prior to oocytes being harvested and analysed (see FIGS. 35 and 36).

FIG. 35 is images showing spindle structure in oocytes from 15 month old C57BL6 females treated with or without NMN for 3 weeks, via addition to drinking water at 2 g/L.

FIG. 36 is a graph showing the number of oocytes collected in PMSG hormonally primed 15 month old wild type mice following treatment with or without NMN, through addition to drinking water (2 g/L) for 4 weeks.

FIG. 37 is a graph showing cell counts of the inner cell mass of blastocysts following in vitro fertilization of oocytes obtained from 8 month old mice treated without NMN, or with NMN through addition to drinking water (2 g/L) for the indicated period of time.

FIG. 38 is a graph showing the proportion of oocytes that did not fertilize, fertilized oocytes that did not develop, blastocysts that did not hatch, and hatched blastocysts, after 5 days following in vitro fertilization of oocytes obtained from 8 month old mice following treatment without NMN, or with NMN by daily gavage (10 mg), or in drinking water (2 g/L).

FIG. 39 is a graph showing the proportion of oocytes that did not fertilize, fertilized oocytes that did not develop, blastocysts that did not hatch, and hatched blastocysts, after 6 days following in vitro fertilization of oocytes obtained from 8 month old mice following treatment without NMN, or with NMN by daily gavage (10 mg), or in drinking water (2 g/L).

### DETAILED DESCRIPTION

The present disclosure relates in one aspect to a method of increasing fertility, or reducing the decline in fertility, in a female subject.

To maintain oocyte reserves in the ovary, oocytes must be arrested at prophase I, which prevents premature meiotic maturation. It is thought that the follicular pool is formed in female mammals during foetal development, and maintained in prophase I arrest in the ovaries until sexual maturity, and released during hormonal cycles. Once released from prophase I, oocytes undergo meiosis, which entails accurate separation and then extrusion of one set of chromosomes into the polar body, to leave behind a euploid oocyte. Both processes are critically dependent upon the essential checkpoint protein BubR1, which regulates the attachment of kinetochores to spindles in both mitotic and meiotic cell types. Levels of BubR1 protein dictate lifespan and biological ageing, with genetic modifications that reduce expression of BubR1 causing an accelerated ageing phenotype, while transgenic over-expression of BubR1 extends lifespan. BubR1 insufficiency causes infertility in mice, while BubR1 levels decline in human oocytes with advancing age. The inventors hypothesised that declining BubR1 levels and subsequent aneuploidy might explain the overall decrease in mammalian female fertility with advanced age, including an increased incidence of spontaneous abortions and offspring born with chromosomal abnormalities.

The inventors have found that increasing SIRT2 levels preserves BubR1 levels in oocytes, and improves fertility.

Sirtuin 2 (SIRT2) is a member of the sirtuin family of NAD⁺-dependent deacylases that mediate the health benefits of dietary restriction.

BubR1 is susceptible to ubiquitination and degradation following acetylation at a key residue, Lys668. Deacetylation of this site by the NAD⁺ dependent deacetylase SIRT2 stabilises BubR1 levels.

As described in the Examples, the inventors have found that over-expression of SIRT2 in aged mice from an exogenously supplied transgene results in increased levels of BubR1 in oocytes, the oocytes produced are of higher quality, and the mice have increased fertility, compared to aged wild-type mice (i.e. mice not expressing the transgene). The inventors have found that by increasing SIRT2 activity and/or expression in aged mice, fertility of the mice can be increased or the rate of decline in fertility reduced.

Thus, in one aspect, the present invention provides a method of increasing fertility of a female subject, the method comprising administering to the subject an effective amount of an agent which elevates SIRT2 activity or SIRT2 expression in the subject. The method increases fertility of the subject, that is, fertility of the subject is increased relative to the fertility of the subject prior to administration of the agent.

As used herein, the “fertility” of a female subject refers to the potential for the oocytes of the subject to be fertilized. An increase in fertility of a female subject is an increase in the likelihood that an oocyte of the subject will be fertilized within a certain time period. An increase in fertility will typically result in a reduced time to pregnancy. The fertility of the female subject may be dependent on a number of factors, including oocyte yield and/or oocyte quality.

Oocyte yield refers to the capacity of a female to produce fertilizable oocytes. Thus, an increase in oocyte yield is an increase in the number of oocytes that are of a quality that is sufficient to be successfully fertilised.

Oocyte quality refers to the capacity of an oocyte to be fertilized, and typically for the fertilized oocyte to proceed to a full term pregnancy.

In one embodiment, an increase in fertility comprises an increase in oocyte quality. In one embodiment, an increase in fertility comprises an increase in oocyte yield. In one embodiment, an increase in fertility comprises an increase in oocyte quality and oocyte yield.

The agent which elevates SIRT2 activity or SIRT2 expression may be administered by any means which permits the agent to elevate SIRT2 activity or SIRT2 expression in the subject.

In some embodiments, the agent may elevate SIRT2 activity or expression in all tissues of the subject. In some embodiments, the agent elevates SIRT2 activity and/or SIRT2 expression in ovarian tissue. Ovarian tissue includes any cells of the ovary including oocytes, oogonial stem cells, follicles. Typically, the agent elevates SIRT2 activity and/or SIRT2 expression in the oocytes.

As SIRT2 is an NAD⁺-dependent deacylase, SIRT2 activity can be increased in a cell by raising NAD⁺ levels, increasing the ratio of NAD⁺ to NADH, and/or increasing production of NAD⁺ in the cell, and/or preventing the breakdown of NAD⁺ by other enzymes.

In one embodiment, the agent which increases SIRT2 activity or SIRT2 expression is an NAD⁺ agonist.

The inventors have found that elevation of NAD⁺ levels through treatment with NAD⁺ agonists in aged female subjects, or female subjects in which the quality of the oocyte is otherwise compromised, such as in chemotherapy, can increase fertility, reduce the rate of decline in fertility, or restore fertility, in the female subjects.

Thus, in one aspect, the present invention provides a method of increasing fertility, reducing the rate of decline in fertility, or restoring fertility, of a female subject, the method comprising administering to the subject an effective amount of an NAD⁺ agonist.

As used herein, an “NAD⁺ agonist” (or “NAD⁺ promoting agent”) is an agent which raises NAD⁺ levels in a cell, and/or increases the ratio of NAD⁺ to NADH in a cell, and/or increases production of NAD⁺ in a cell.

The NAD⁺ agonist may be administered by any means which permits the NAD⁺ agonist to raise NAD⁺ levels in cells of the subject, and/or increase ratio of NAD⁺ to NADH in cells of the subject and/or increase production of NAD⁺ in cells of the subject.

In one embodiment, the NAD⁺ agonist is an agent which raises NAD⁺ levels in a cell, e.g. an oocyte. An agent which raises NAD⁺ levels in a cell increases the amount of NAD⁺ in the cell relative to the amount of NAD⁺ in the cell prior to contact with the agent.

In one embodiment, the NAD⁺ agonist is an agent which increases the ratio of NAD⁺ to NADH in a cell, e.g. an oocyte. An agent which raises the ratio of NAD⁺ to NADH in a cell increases the ratio of NAD⁺ to NADH in the cell relative to the ratio of NAD⁺ to NADH in the cell prior to contact with the agent.

In one embodiment, the NAD⁺ agonist is an agent which increases production of NAD⁺ in a cell, e.g. an oocyte. An agent which increases production of NAD⁺ in a cell increases the production of NAD⁺ in the cell relative to the production of NAD⁺ in the cell prior to contact with the agent.

In one embodiment, the NAD⁺ agonist raises NAD⁺ levels in an oocyte and increases the ratio of NAD⁺ to NADH in an oocyte. In one embodiment, the NAD⁺ agonist raises NAD⁺ levels in an oocyte, increases the ratio of NAD⁺ to NADH in the oocyte and increases the rate of production of NAD⁺ in the oocyte. In one embodiment, the NAD⁺ agonist raises NAD⁺ levels in an oocyte and increases production of NAD⁺ in the oocyte.

Methods for determining the amount of NAD⁺ in a cell, the ratio of NAD⁺ to NADH in a cell, and the production of NAD⁺ in a cell, are known in the art and are described in, for example, Schwartz et al. (1974) J. Biol. Chem. 249:4138-4143; Sauve and Schramm (2003) Biochemistry 42(31):9249-9256; Yamada et al. (2006) Analytical Biochemistry 352:282-285, or can be determined using commercially available kits such as, for example, NAD/NADH-Glo Assay (Promega Inc.) or NAD/NADH Quantitation Colorimetric Kit (BioVision Inc.).

In one form, the NAD⁺ agonist reduces breakdown of NAD⁺ in a cell, e.g. an oocyte, thereby raising the NAD⁺ levels in the cell. An example of an agent which reduces the breakdown of NAD⁺ in cells, including oocytes, is a CD38 inhibitor. CD38 is an enzyme which catalyzes the synthesis and hydrolysis of cyclic ADP-ribose from NAD⁺ and ADP-ribose. CD38 reduces NAD⁺ levels in the cell by converting NAD⁺ to cyclic ADP-ribose. Thus, in one embodiment, the NAD⁺ agonist is a CD38 inhibitor.

As used herein, a “CD38 inhibitor” is an agent which reduces or eliminates the biological activity of CD38. The biological activity of CD38 may be reduced or eliminated by inhibiting enzyme function, or by inhibiting expression of CD38 at the level of gene expression and enzyme production. “Inhibiting” is intended to refer to reducing or eliminating, and contemplates both partial and complete reduction or elimination.

In one embodiment, the CD38 inhibitor is an inhibitor of CD38 enzyme function. An inhibitor of CD38 enzyme function is an agent that blocks or reduces the enzymatic activity of CD38.

In one embodiment, the inhibitor of CD38 enzyme function is a compound of formula I:

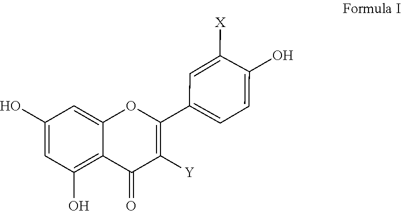

wherein:
X is H or OH; and
Y is H or OH;
or a pharmaceutically acceptable salt, derivative or prodrug thereof.

In one embodiment, X and Y are both H.

An example of an inhibitor of CD38 enzyme function is apigenin, or a pharmaceutically acceptable salt, derivative or prodrug thereof. Apigenin (5,7-dihydroxy-2-(4-hydroxyphenyl)-4H-1-benzopyran-4-one), also known as 4′,5,7-trihydroxyflavone, is an isoflavone found in plants, including fruits and vegetables, such as parsley, celery and chamomile. Apigenin has the following structure:

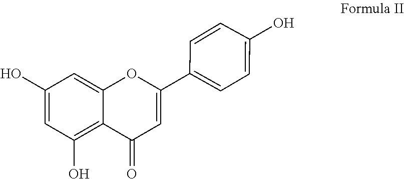 [SMILES: O=c1cc(-c2ccc(O)cc2)oc2cc(O)cc(O)c12]

Another example of an inhibitor of CD38 enzyme function is quercetin, or a pharmaceutically acceptable salt, derivative or prodrug thereof. Quercetin [2-(3,4-dihydroxyphenyl)-3,5,7-trihydroxy-4H-chromen-4-one]) is an isoflavone found in plants, including fruits, vegetables, leaves and grains. Quercetin has the following structure:

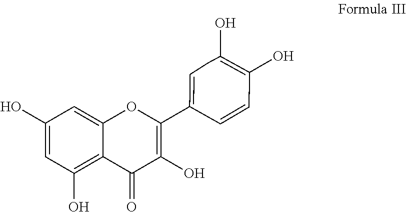 [SMILES: O=c1c(O)c(C2=CCC(O)=C(O)C=C2)oc2cc(O)cc(O)c12]

Both apigenin and quercetin have been shown to be inhibitors of CD38 activity in vitro (Esande et al. (2013) Diabetes, 1084-1093).

Isoflavones (such as apigenin or quercetin) are typically administered in isolated form. By “isolated” it is meant that the isoflavone has undergone at least one purification step. When the inhibitor of CD38 enzyme function is an isoflavone, the inhibitor is conveniently administered in a composition comprising at least 10% w/v inhibitor, at least 20% w/v inhibitor, at least 30% w/v inhibitor, at least 40% w/v inhibitor, at least 50% w/v inhibitor, at least 60% w/v inhibitor, at least 70% w/v inhibitor, at least 80% w/v inhibitor, at least 90% w/v inhibitor, at least 95% w/v inhibitor, or at least 98% w/v inhibitor. In one embodiment, the inhibitor is in a biologically pure form (i.e. substantially free of other biologically active compounds). Methods for isolation of biologically pure forms of isoflavones such as apigenin and quercetin are known in the art. Biologically pure apigenin and quercetin is also commercially available from, for example, Sigma Chemical Company (St. Louis) (Cat. No. A3145 and Cat. No. Q4951), or Indofine Chemical Company (Cat. No. A-002).

In some embodiments, the CD38 inhibitor is a pharmaceutically acceptable salt or pro-drug form of the inhibitor of CD38 enzyme function, such as a pharmaceutically acceptable salt or prodrug of apigenin or quercetin. The term “prodrug” is used herein in its broadest sense to include those compounds which are converted in vivo to the active form of the drug. Use of the prodrug strategy may optimise the delivery of the NAD⁺ agonist to its site of action.

In one embodiment, the pro-drug of the inhibitor of CD38 enzyme function is an ester or an imine of the inhibitor.

In one embodiment, the NAD⁺ agonist is apigenin, or a pharmaceutically acceptable salt, derivative or prodrug thereof.

In another embodiment, the CD38 inhibitor is an inhibitor of CD38 gene expression or enzyme production. An inhibitor of CD38 gene expression or enzyme production is an agent that blocks or reduces transcription or translation of the CD38 gene. Inhibition of CD38 gene expression or enzyme production may be, for example, by RNA interference (RNAi) (e.g. siRNA, shRNA), antisense nucleic acid, locked nucleic acid (LNA), DNAzymes, or ribozymes, which target CD38 mRNA transcripts, by genome editing technologies such as Zinc finger nucleases (ZFN), Transcription Activator-Like effector Nucleases (TALENS), Clustered regular Interspaced Short Palindromic Repeats (CRISPR), or engineered meganuclease reengineered homing nuclease, which target the CD38 gene. “RNAi” refers to a nucleic acid that forms a double stranded RNA, which double stranded RNA has the ability to reduce or inhibit expression of a target gene when the siRNA is present in the same cell as the gene or target gene. “shRNA” or “short hairpin RNA” refers to a nucleic acid that forms a double stranded RNA with a tight hairpin loop, which has the ability to reduce or inhibit expression of a gene or target gene. An “antisense” polynucleotide is a polynucleotide that is substantially complementary to a target polynucleotide and has the ability to specifically hybridize to the target polynucleotide to decrease expression of a target gene. Ribozymes and DNAzymes are catalytic RNA and DNA molecules, respectively, which hybridise to and cleave a target sequence to thereby reduce or inhibit expression of the target gene. General methods of using antisense, ribozyme, DNAzyme and RNAi technology, to control gene expression, are known in the art. Genome editing uses artificially engineered nucleases to create specific double strand breaks at desired locations in the genome, and harnesses the cells endogenous mechanisms to repair the breaks. Methods for silencing genes using genome editing technologies are described in, for example, Tan et al. (2012) Precision editing of large animal genomes, Adv. Genet. 80: 37-97; de Souza (2011) Primer: Genome editing with engineered nucleases, Nat. Meth. 9(1) 27-27; Smith et al. (2006) A combinatorial approach to create artificial homing endonucleases cleaving chosen sequences, Nucleic Acids Research 34: 22, e149; Umov et al. (2010) Nat. Rev. Genet. 11(9): 636-646. Inhibition of CD38 expression using iRNA is described in, for example, Escande et al. (2013) Diabetes, 62: 1084-1093.

In another embodiment, the NAD⁺ agonist is an agent which promotes synthesis of NAD⁺ in a cell, e.g. an oocyte, thereby raising NAD⁺ levels in the cell. An example of an agent which promotes synthesis of NAD⁺ is an NAD⁺ precursor.

Thus, in one aspect, the present invention provides a method of increasing fertility, reducing the rate of decline in fertility, or restoring fertility, of a female subject, the method comprising administering to the subject an effective amount of an NAD⁺ precursor.

As used herein, an “NAD⁺ precursor” is an intermediate of NAD⁺ synthesis which does not inhibit sirtuin activity. Examples of NAD⁺ precursors include nicotinamide mononucleotide (NMN), nicotinamide riboside (NR), nicotinic acid riboside (NaR), ester derivatives of nicotinic acid riboside, nicotinic acid (niacin), ester derivatives of nicotinic acid, nicotinic acid mononucleotide (NaMN), ester derivatives of nicotinic acid mononucleotide, nicotinic acid adenine dinucleotide (NaAD), nicotinic acid adenine dinucleotide (NAAD), 5-phospho-α-D-ribosyl-1-pyrophosphate (PPRP), or a pharmaceutically acceptable salt, derivative or prodrug thereof.

In one embodiment, the NAD⁺ agonist is NMN or a pharmaceutically acceptable salt, derivative or prodrug thereof, NR or a pharmaceutically acceptable salt, derivative or prodrug thereof, or NAAD or a pharmaceutically acceptable salt, derivative or prodrug thereof.

In one embodiment, the NAD⁺ agonist is NMN or a pharmaceutically acceptable salt, derivative or prodrug thereof.

In one embodiment, the NAD⁺ agonist is NR or a pharmaceutically acceptable salt, derivative or prodrug thereof. Examples of derivatives of NR and methods for their production, are described in, for example, U.S. Pat. No. 8,106,184.

In one embodiment, the NAD⁺ agonist is NAAD or a pharmaceutically acceptable salt, derivative or prodrug thereof.

In one embodiment, the NAD⁺ agonist is NaMN or a pharmaceutically acceptable salt, derivative or prodrug thereof.

In one embodiment, the NAD⁺ agonist is NaR or a pharmaceutically acceptable salt, derivative or prodrug thereof.

In some embodiments, the NAD⁺ agonist is supplemented into the food or drinking water of a companion, racing, or agricultural animal breed.

In embodiments in which an oocyte is injected or permeabilised to introduced the NAD⁺ agonist, the NAD⁺ agonist may in some embodiments be NAD⁺, or derivative or prodrug thereof.

In another embodiment, NAD⁺ levels may be raised by reducing inhibition of translation of the NAD⁺ biosynthetic enzymes NAMPT, NMNAT1, NMNAT2, and NMNAT3. Inhibition of translation of the NAD⁺ biosynthetic enzymes NAMPT, NMNAT1, NMNAT2, and NMNAT3 is mediated by endogenous micro RNA (miRNA) that target NAMPT, NMNAT1, NMNAT2, and NMNAT3. Thus, NAD⁺ levels may be raised in the endothelial cell by inhibiting the activity of endogenous miRNA which targets NAMPT, NMNAT1, NMNAT2, and NMNAT3. Accordingly, in one embodiment, the NAD⁺ agonist is an NAMPT, NMNAT1, NMNAT2, and/or NMNAT3 miRNA antagonist. As used herein, a “NAMPT, NMNAT1, NMNAT2, and/or NMNAT3 miRNA antagonist” is an agent which inhibits the activity of miRNA that inhibits translation of any one or more of NAMPT, NMNAT1, NMNAT2, and NMNAT3. The NAMPT, NMNAT1, NMNAT2, and/or NMNAT3 miRNA antagonist may act by inhibiting NAMPT, NMNAT1, NMNAT2, and/or NMNAT3 miRNA through, for example, RNA interference (RNAi) (e.g. siRNA, shRNA), antisense nucleic acid, locked nucleic acid (LNA), DNAzymes, or ribozymes, which target miRNAs that target NAMPT, NMNAT1, NMNAT2, and/or NMNAT3, or by genome editing technologies such as Zinc finger nucleases (ZFN), Transcription Activator-Like effector Nucleases (TALENS), Clustered regular Interspaced Short Palindromic Repeats (CRISPR), or engineered meganuclease reengineered homing nuclease, which target the DNA sequences which encode the miRNAs that target NAMPT, NMNAT1, NMNAT2, and/or NMNAT3. Activation domains may be targeted to the genes of NAD biosynthetic genes (e.g. NAMPT, NMNAT1, NMNAT2, and/or NMNAT3) to increase gene expression using CRISPR-directed heterologous regulatory domains (e.g. VP16 or VP64).

In another embodiment, the NAD⁺ levels may be raised by increasing expression of NAMPT, NMNAT1, NMNAT2, and/or NMNAT3. Expression of NAMPT, NMNAT1, NMNAT2, and/or NMNAT3 can be increased by administering an agent comprising a transgene expressing NAMPT, NMNAT1, NMNAT2, and/or NMNAT3. Accordingly, in some embodiments, the NAD⁺ agonist is an agent comprising a transgene expressing NAMPT, NMNAT1, NMNAT2, and/or NMNAT3. In one embodiment, the transgene expresses NMNAT1.

As described in the Examples, the inventors have found that over-expression of NMNAT1 in oocytes of aged mice from an exogenously supplied transgene results in increased production of oocytes in the mice compared to aged mice not expressing the transgene.

In another embodiment, NAD⁺ levels in a cell, e.g. an oocyte, may be raised by contacting the cell with an NAD⁺ agonist which enhances the enzymatic activity of NAD⁺ biosynthetic enzymes, such as the NAD⁺ biosynthetic enzymes NAMPT, NMNAT1, NMNAT2, and/or NMNAT3 or PNC1 from other species such as yeast, flies or plants. Accordingly, in some embodiments, the NAD+ agonist is an agent which enhances the enzymatic activity of NAD⁺ biosynthetic enzymes, such as the NAD⁺ biosynthetic enzymes NAMPT, NMNAT1, NMNAT2, and/or NMNAT3 or PNC1 from other species such as yeast, flies or plants. For example, P7C3 enhances activity of NAMPT in vitro, thereby increasing the level of intracellular NAD⁺ (Wang et al. (2014) Cell, 158(6):1324-1334). P7C3 has the following structure:

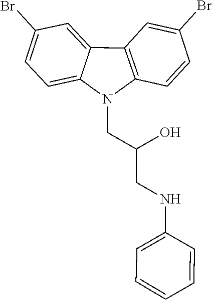 [SMILES: OC(CNc1ccccc1)Cn1c2ccc(Br)cc2c2cc(Br)ccc21]

The enzymatic activity of NAD⁺ biosynthetic enzymes, such as NAMPT, NMNAT1, NMNAT2, and/or NMNAT3, may be enhanced by introducing into cells of the subject nucleic acid which expresses one or more of the NAD⁺ biosynthetic enzymes in cell of the subject (e.g. oocytes).

In one embodiment, the NAD⁺ agonist is an agent which increases the ratio of NAD⁺ to NADH in the cell relative to the ratio of NAD⁺ to NADH in the cell prior to contact with the NAD⁺ agonist. For example, the ratio of the amount of NAD⁺ to NADH may be increased by contacting the cell with an NAD⁺ agonist which activates an enzyme that converts NADH to NAD⁺. For example, β-lapachone (3,4-dihydro-2,2-dimethyl-2H-napthol[1,2-b]pyran-5,6-dione) activates the enzyme NADH:quinone oxidoreductase (NQ01) which catalyses the reduction of quinones to hydroquinones by utilizing NADH as an electron donor, with a consequent increase in the ratio of NAD⁺ to NADH.

Accordingly, in one embodiment, the NAD⁺ agonist is an activator of NQ01, such as lapachone, or a pharmaceutically acceptable salt, derivative or prodrug thereof.

As described in the Examples, the inventors have found that expression of a SIRT2 transgene under the control of a constitutive promoter in a female subject results in increased fertility.

In one embodiment, the agent which elevates expression of SIRT 2 comprises a nucleic acid that is capable of expressing SIRT2 in a subject. A nucleic acid that is capable of expressing SIRT2 in a subject may comprise the coding sequence of SIRT2 operably linked to regulatory sequence which operate together to express a protein encoded by the coding sequence. “Coding sequence” refers to a DNA or RNA sequence that codes for a specific amino acid sequence. It may constitute an “uninterrupted coding sequence”, i.e., lacking an intron, such as in a cDNA, or it may include one or more introns bounded by appropriate splice junctions. An example of human SIRT2 coding sequence is the nucleotide sequence from nucleotide 257 to 1315 of Genbank accession no. BC003547.1 (SEQ ID NO: 1). A “regulatory sequence” is a nucleotide sequence located upstream (5′ non-coding sequences), within, or downstream (3′ non-coding sequences) of a coding sequence, and which influences the transcription, RNA processing or stability, or translation of the associated coding sequence. Regulatory sequences are known in the art and may include, for example, transcriptional regulatory sequences such as promoters, enhancers translation leader sequences, introns, and polyadenylation signal sequences. The coding sequence is typically operably linked to a promoter. A promoter is a DNA region capable under certain conditions of binding RNA polymerase and initiating transcription of a coding sequence usually located downstream (in the 3′ direction) from the promoter. The coding sequence may also be operably linked to termination signals. The expression cassette may also include sequences required for proper translation of the coding sequence. The coding sequence may be under the control of a constitutive promoter or a regulatable promoter that initiates transcription in, for example, oocytes of the ovarian tissue. For example, the SIRT2 coding sequence may be operably linked to a promoter which is not native to the SIRT2 gene, such as a promoter that expresses the coding sequence in, or is inducible in, oocytes. Examples of suitable promoters include Oogl, Zp3, Msy2 and others.

A nucleic acid encoding a protein (coding sequence) is operably linked to a regulatory sequence when it is arranged relative to the regulatory sequence to permit expression of the protein in a cell. For instance, a promoter is operatively linked to a coding region if the promoter helps initiate transcription of the coding sequence.

As used herein, “expression” of a nucleic acid sequence refers to the transcription and translation of a nucleic acid sequence comprising a coding sequence to produce the polypeptide encoded by the coding sequence.

The nucleic acid sequence encoding SIRT2 may be inserted into an appropriate vector sequence. The term “vector” refers to a nucleic acid sequence suitable for transferring genes into a host cell. The term “vector” includes plasmids, cosmids, naked DNA, viral vectors, etc. In one embodiment, the vector is a plasmid vector. A plasmid vector is a double stranded circular DNA molecule into which additional sequence may be inserted. The plasmid may be an expression vector. Plasmids and expression vectors are known in the art and described in, for example, Sambrook et al. Molecular Cloning: A Laboratory Manual, 4^{th} Ed. Vol. 1-3, Cold Spring Harbor, N.Y. (2012).

In some embodiments, the vector is a viral vector. Viral vectors comprise viral sequence which permits, depending on the viral vector, viral particle production and/or integration into the host cell genome and/or viral replication. Viral vectors which can be utilized with the methods and compositions described herein include any viral vector which is capable of introducing a nucleic acid into endothelial cells, such as endothelial cells of skeletal muscle. Examples of viral vectors include adenovirus vectors; lentiviral vectors; adeno-associated viral vectors; Rabiesvirus vectors; Herpes Simplex viral vectors; SV40; polyoma viral vectors; poxvirus vector.

In some embodiments, the nucleic acid comprises a coding sequence which encodes a protein or RNA which causes the activity of SIRT2 or expression of SIRT2 to be increased in cells (e.g. in oocytes) of the female subject. In various embodiments, the coding sequence encodes:
(a) SIRT2 protein;
(b) one or more NAD⁺ biosynthetic enzymes, or
(c) an NAMPT, NMNAT1, NMNAT2, and/or NMNAT3 miRNA antagonist.

In one embodiment, the coding sequence encodes SIRT2. Examples of SIRT2 amino acid sequence include Genbank accession numbers NP_071877.3 (mouse) (SEQ ID NO:2), AAK51133.1, (human) (SEQ ID NO: 3), and NP_001008369.1 (rat) (SEQ ID NO: 4).

In one embodiment, the coding sequence encodes a protein or RNA which causes NAD⁺ levels to be increased in cells (e.g. oocytes) of a female subject. In one embodiment, the coding sequence encodes one or more NAD⁺ biosynthetic enzymes selected from the group consisting of NAMPT, NMNAT1, NMNAT2, and NMNAT3. Examples of the amino acid sequence of NAMPT is Genbank accession numbers NP_005737.1 (human) (SEQ ID NO: 5), NP_067499.2 (mouse) (SEQ ID NO: 6), XP_022261566.1 (dog) (SEQ ID NO: 7); examples of the amino acid sequence of NMNAT1 is Genbank accession numbers AAH14943.1 (human) (SEQ ID NO: 8), NP_597679.1 (mouse) (SEQ ID NO: 9), XP_005620579.1 (dog) (SEQ ID NO: 10); examples of the amino acid sequence of NMNAT2 is Genbank accession numbers NP_055854.1 (human) (SEQ ID NO: 11), NP_780669.1 (mouse) (SEQ ID NO: 12), XP_022276670.1 (dog) (SEQ ID NO: 13); examples of the amino acid sequence of NMNAT3 is AAH36218.1 (human) (SEQ ID NO: 14), (mouse) (SEQ ID NO: 15), (XP_022264401.1) (dog) (SEQ ID NO: 16).

In one embodiment, the coding sequence encodes a NAMPT, NMNAT1, NMNAT2, and/or NMNAT3 miRNA antagonist.

In one embodiment, the coding sequence which encodes: SIRT2 protein; one or more NAD⁺ biosynthetic enzymes, or the NAMPT, NMNAT1, NMNAT2, and/or NMNAT3 miRNA antagonist, is operably linked to a promoter which expresses the coding sequence in cells of the subject, such as in oocytes. In one embodiment, the promoter is selected from the group consisting of Oog1, Zp3, and Msy2.

The nucleic acid may be incorporated into a viral vector for administering to the subject. Accordingly, in one aspect, there is provided a viral vector, wherein the viral vector comprises nucleic acid which comprises coding sequence which encodes a protein or RNA which causes the activity of SIRT2 or expression of SIRT2 to be increased in cells of a female subject, (e.g., occytes). In various embodiments, the coding sequence encodes:
(a) SIRT2 protein;
(b) one or more NAD⁺ biosynthetic enzymes, or
(c) an NAMPT, NMNAT1, NMNAT2, and/or NMNAT3 miRNA antagonist.

In one embodiment, the coding sequence is operably linked to a promoter which expresses the coding sequence in, or is inducible in, oocytes. In one embodiment, the promoter is selected from the group consisting of Oogl, Zp3, Msy2. Typical viral vectors are as mentioned above, and include adenovirus vectors; lentiviral vectors; adeno-associated viral vectors; Rabiesvirus vectors; Herpes Simplex viral vectors; SV40; polyoma viral vectors; poxvirus vector.

In one embodiment, the viral vector is an adeno-associated viral (AAV) vector. In one embodiment, the AAV vector is a serotype selected from the group consisting of AAV1, AAV2, AAV3, AAV4, AAV5, AAV6, AAV6.2, AAV7, AAV8, and AAV9 vector or variants thereof. The use of recombinant AAV vectors for introducing nucleic acids into cells is known in the art and described in, for example, US20160038613; Grieger and Samulski (2005) Adeno-associated virus as a gene therapy vector: vector development, production and clinical applications, Advances in Biochemical Engineering/Biotechnology 99: 119-145; Methods for the production of recombinant AAV are known in the art and described in, for example, Harasta et al (2015) Neuropsychopharmacology 40: 1969-1978.

Viral vectors are typically packaged into viral particles using methods known in the art. The viral particles may then be used to transfer the nucleic acid to a subject. Thus, another aspect provides a virus comprising a viral vector as described herein.
In various aspects, there is provided a method of
(a) increasing fertility in a female subject;
(b) increasing oocyte yield in a female subject;
(c) increasing oocyte quality in a female subject;
(d) improving in vitro fertilisation (IVF) success rates;
(e) providing prophylaxis against infertility in patients receiving chemotherapy or radiotherapy;
(f) restoring fertility following chemotherapy;
(g) treating or preventing infertility in a female subject;
(h) reducing aneuploidy of an oocyte;
(i) reducing aneuploidy of an oocyte in a female subject;
(j) reducing the rate at which BubR1 activity decreases with age in an oocyte of a female subject;
(k) increasing fertilisation potential of an oocyte;
(l) fertilizing an oocyte in vitro;
(m) promoting regeneration of ovarian follicles in an adult female subject,

comprising administering an effective amount of the virus described herein.

As used herein, the term “subject” refers to an animal, and the term “female subject” refers to a subject (i.e. an animal) that is genetically female and has at least one ovary. In one embodiment, the animal is a mammal. In one embodiment, the mammal is a human. In one embodiment the mammal is a non-human.

A non-human mammal may, for example, be a primate, sheep, cow, horse, donkey, pig, dog, cat, mouse, rabbit, rat, guinea pig, hamster, fox, deer, or monkey. In some embodiments, the mammal is a stud animal, such as a cow, horse, pig or sheep. In some embodiments, the mammal is an agricultural animal, such as a dairy cow, or a pig, or a racing animal, such as a horse or greyhound, or a companion animal, such as a dog or cat.

Although the present invention is exemplified using a murine model, the method of the present invention may be applied to other species.

As further described in the Examples, the inventors have found that administering of an NAD⁺ agonist, such as the NAD⁺ precursor NMN, to aged mice, or mice treated with a chemotherapeutic agent such as doxorubicin or cisplatin:
(a) increases fertility;
(b) increases oocyte yield;
(c) increases oocyte quality;
(d) restores or preserves fertility;
(e) reduces oxidative damage to oocytes;
(f) improves spindle assembly in oocytes;
(g) reduces aneuploidy in oocytes; and
(h) reduces the rate at which BubR1 activity decreases.

Accordingly, in various aspects, the present invention provides a method of:
(a) increasing fertility in a female subject;
(b) increasing oocyte yield in a female subject;
(c) increasing oocyte quality in a female subject;
(d) improving in vitro fertilisation (IVF) success rates;
(e) providing prophylaxis against infertility in patients receiving chemotherapy or radiotherapy;
(f) restoring fertility following chemotherapy;
(g) treating or preventing infertility in a female subject;
(h) reducing aneuploidy of an oocyte;
(i) reducing aneuploidy of an oocyte in a female subject;
(j) reducing the rate at which BubR1 activity decreases with age in an oocyte of a female subject;
(k) increasing fertilisation potential of an oocyte;
(l) fertilizing an oocyte in vitro;
(m) promoting regeneration of ovarian follicles in an adult female subject;
(n) improving or enhancing the ability of an oocyte to form blastocysts during IVF,

comprising administering to the subject, or introducing into an oocyte, an effective amount of an NAD⁺ agonist.

In various aspects, the present invention provides a method of:
(a) increasing fertility in a female subject;
(b) increasing oocyte yield in a female subject;
(c) increasing oocyte quality in a female subject;
(d) improving in vitro fertilisation (IVF) success rates;
(e) providing prophylaxis against infertility in patients receiving chemotherapy or radiotherapy;
(f) restoring fertility following chemotherapy;
(g) treating or preventing infertility in a female subject;
(h) reducing aneuploidy of an oocyte;
(i) reducing aneuploidy of an oocyte in a female subject;
(j) reducing the rate at which BubR1 activity decreases with age in an oocyte of a female subject;
(k) increasing fertilisation potential of an oocyte;
(l) fertilizing an oocyte in vitro;
(m) promoting regeneration of ovarian follicles in an adult female subject;
(n) improving or enhancing the ability of an oocyte to form blastocysts during IVF,

comprising administering to the subject, or introducing into an oocyte, an effective amount of an NAD⁺ precursor.

In various further aspects, the present invention provides an NAD⁺ precursor for use in:
(a) increasing fertility in a female subject;
(b) increasing oocyte yield in a female subject;
(c) increasing oocyte quality in a female subject;
(d) improving in vitro fertilisation (IVF) success rates;
(e) providing prophylaxis against infertility in patients receiving chemotherapy or radiotherapy;
(f) restoring fertility following chemotherapy;
(g) treating or preventing infertility in a female subject;
(h) reducing aneuploidy of an oocyte;
(i) reducing aneuploidy of an oocyte in a female subject;
(j) reducing the rate at which BubR1 activity decreases with age in an oocyte of a female subject;
(k) increasing fertilisation potential of an oocyte;
(l) fertilizing an oocyte in vitro;
(m) promoting regeneration of ovarian follicles in an adult female subject;

(n) improving or enhancing the ability of an oocyte to form blastocysts during IVF.

In various embodiments, the NAD⁺ precursor is:
(a) NMN or a pharmaceutically acceptable salt, derivative or prodrug thereof;
(b) NR or a pharmaceutically acceptable salt, derivative or prodrug thereof;
(c) NAAD or a pharmaceutically acceptable salt, derivative or prodrug thereof;
(d) NaR or a pharmaceutically acceptable salt, derivative or prodrug thereof;
(e) nicotinic acid (niacin), an ester derivative of nicotinic acid, or a pharmaceutically acceptable salt, derivative or prodrug thereof;
(f) NaMN or a pharmaceutically acceptable salt, derivative or prodrug thereof;
(g) PPRP or a pharmaceutically acceptable salt, derivative or prodrug thereof.

In one embodiment, the NAD+ precursor is NMN or a pharmaceutically acceptable salt, derivative or prodrug thereof.

The inventors have shown that by administering to a female subject an NAD⁺ agonist, such as NMN, female fertility can be preserved during ageing, or during insults which adversely affect the quality of oocytes, such as chemotherapy. Further, the inventors have shown that by administering to a female subject an NAD⁺ agonist, such as NMN, female fertility can be restored in ageing female subjects, or in female subjects in which the quality of the oocyte is compromised from insults which adversely affect the quality of the oocyte, such as chemotherapy.

Accordingly, in one embodiment, the present invention provides a method of treating or preventing infertility in a female subject suffering from a decline in fertility, or at risk of suffering from a decline in fertility, or suffering from infertility, such as an aged female subject, or a female subject who has received, is receiving, or is to receive, an insult which adversely affects the quality of the oocytes of the subject, or a female subject who has an underlying predisposition to infertility. The method comprises administering to the female subject an effective amount of an agent which elevates SIRT2 activity or SIRT2 expression in the subject. In one embodiment, the agent which elevates SIRT2 activity or SIRT2 expression in the subject is an NAD⁺ agonist. Typically, the agent which elevates SIRT2 activity or SIRT2 expression in the subject is an NAD⁺ precursor.

The subject may be any female subject with at least one ovary. In some embodiments the subject is an aged subject. An aged subject is a subject is a subject that is at an age in which the quality of the oocytes is in decline. Typically, the subject is an aged human. The aged human subject may have an age that is greater than 30 years, greater than 35 years, or greater than 40 years, more typically in the range of from 30 to 55 years, still more typically 35 to 50 years. It will be appreciated that what is considered middle aged and aged will depend on the species of the subject and can be readily determined by those skilled in the art.

In some embodiments, the subject is a subject who has received an insult which adversely affects the quality of their oocytes. Examples of insults which may adversely affect the quality of a subject's oocytes include chemotherapeutic agents, radiation exposure such as in radiotherapy or x-ray exposure, pesticides, fungicides, herbicides, cigarette smoke, marijuana, cocaine, or diets that cause obesity. Examples of chemotherapeutic agents which may adversely affect the quality of oocytes in a subject include mechlorethamine, ifosfamide, melphalan, chlorambucil, cyclophosphamide, streptozocin, carmustine, lomustine, busulfan, dacarbazine, temozolomide, thiotepa, altreamine; cisplatin, doxorubicin, carboplatin, and procarbazine.

In some embodiments, the subject is pre-menopausal. In some embodiments, the subject is post-menopausal. In some embodiments, the subject is a pre-pubertal child. In such embodiments, the subject may be treated to prevent or improve the fertility of the subject after puberty, e.g. in a pre-pubertal subject diagnosed as suffering from a condition likely to lead to low fertility or infertility, or who has been, or is likely to be, exposed to an insult, such as chemotherapy or radiotherapy, that is likely to prevent or reduce future fertility.

In some embodiments, the subject is an overweight or obese subject.

In some embodiments, the subject is suffering from hormonal disturbances, such as polycystic ovarian syndrome.

In some embodiments, the subject has an underlying predisposition to infertility, such as premature ovarian failure.

As used herein, “treating” means affecting a subject, tissue or cell to obtain a desired pharmacological and/or physiological effect and includes inhibiting the condition, i.e. arresting its development; or relieving or ameliorating the effects of the condition i.e., cause reversal or regression of the effects of the condition. As used herein, “preventing” means preventing a condition from occurring in a cell or subject that may be at risk of having the condition, but does not necessarily mean that condition will not eventually develop, or that a subject will not eventually develop a condition. Preventing includes delaying the onset of a condition in a cell or subject.

The term “effective amount” refers to the amount of the compound that will elicit the biological or medical response of a tissue, system, animal or human that is being sought by the researcher, veterinarian, medical doctor or other clinician.

The agent which elevates SIRT2 activity or expression, such as an NAD⁺ agonist, may be administered or introduced as a pharmaceutical composition comprising the agent, and a pharmaceutically acceptable carrier. A “pharmaceutically acceptable carrier” is a carrier that it is compatible with the other ingredients of the composition and is not deleterious to a subject, or in cases of in vitro applications, the oocyte. The compositions may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavours, etc.) according to techniques such as those well known in the art of pharmaceutical formulation (See, for example, Remington: The Science and Practice of Pharmacy, 21st Ed., 2005, Lippincott Williams & Wilkins).

In some embodiments, the carrier is a synthetic (non-naturally occurring) carrier.

For in vivo applications, the agent which elevates SIRT2 activity or expression (e.g. an NAD⁺ agonist) may be administered by any means which permits the agent to elevate SIRT2 activity or expression in the subject. In some embodiments, the agent may be administered orally, such as in the form of tablets, capsules, granules or powders; sublingually; buccally; parenterally, such as by subcutaneous, intravenous, intramuscular, intra(trans)dermal, intraperitoneal, or intracisternal injection or infusion techniques (e.g., as sterile injectable aqueous or non-aqueous solutions or suspensions), or in the form of an implant; nasally such as by inhalation spray or insufflation; in dosage unit formulations containing non-toxic, pharmaceutically acceptable vehicles or diluents. The agent may, for example, be administered in a form suitable for immediate release or extended release. Immediate release or extended release may be achieved by the use of suitable pharmaceutical compositions comprising the agent. Typically, the agent is administered orally.

The pharmaceutical compositions for in vivo administration may conveniently be presented in dosage unit form and may be prepared by any of the methods well known in the art of pharmacy. These methods generally include the step of bringing the active agent (e.g. the NAD⁺ agonist) into association with the carrier which constitutes one or more accessory ingredients. In general, the pharmaceutical compositions are prepared by uniformly and intimately bringing the compound into association with a liquid carrier or a finely divided solid carrier or both, and then, if necessary, shaping the product into the desired formulation. In the pharmaceutical composition the active compound is included in an amount sufficient to produce the desired effect.

The pharmaceutical compositions for in vivo applications may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents such as sweetening agents, flavouring agents, colouring agents and preserving agents, e.g. to provide pharmaceutically stable and palatable preparations. Tablets containing one or more NAD⁺ agonist, may be prepared in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release.

Formulations for oral use may also be presented as hard gelatin capsules wherein the agent which elevates SIRT2 activity or expression is mixed with an inert solid diluent, for example, calcium carbonate, calcium phosphate or kaolin, or as soft gelatin capsules wherein the agent is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the agent which elevates SIRT2 activity or expression (e.g. the NAD⁺ agonist) in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavouring and colouring agents.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectable formulations.

The agent which elevates SIRT2 activity or expression (e.g. the NAD⁺ agonist) can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolisable lipid capable of forming liposomes can be used. The present compositions in liposome form can contain, in addition to a compound of the present invention, stabilizers, preservatives, excipients and the like. The preferred lipids are the phospholipids and phosphatidyl cholines, both natural and synthetic. Methods to form liposomes are known in the art.

The agent which elevates SIRT2 activity or expression (e.g. an NAD+ agonist) can also be administered in the form of a crystalline product for superior stabilization and purity.

It will be understood that the specific dose level and frequency of dosage for any particular subject may be varied and will depend upon a variety of factors including the activity of the specific compound employed, the metabolic stability and length of action of that compound, the age, body weight, general health, diet, mode and time of administration, the rate of excretion, drug combinations, and the severity of the particular condition.

For in vitro applications comprising introducing an agent into an oocyte, such as in ART, the agent may be introduced into oocyte by any means which results in agent having an effect on the oocyte. Typically, the agent, or a composition comprising the agent, is contacted with the oocyte under conditions whereby the agent enters the cell. For example, if the agent is able to cross the cell membrane, the agent may be contacted with the oocyte by, for example, incubating the oocyte in medium containing the agent, for example, during in vitro maturation (IVM) from follicles, or during IVF. In some embodiment, the agent may be transfected or injected into the oocyte, for example, during intracytoplasmic sperm injection (ICSI). Typically, the agent is introduced into the oocyte in an aqueous composition. In one embodiment, the agent is introduced into oocytes by dissolving or dispersing the agent in the same solution used to inject sperm into an oocyte during intracytoplasmic sperm injection (ICSI). Methods for ICSI and suitable solutions for such methods are disclosed in, for example, Kang et al. (2015) Clin. Exp. Reprod. Med. 42(2):45-50.

A further aspect provides a method of fertilizing an oocyte, comprising injecting an oocyte with a sperm and an NAD⁺ agonist, such as NAD⁺ or an NAD⁺ precursor. The NAD⁺ agonist may be introduced into the oocyte before, during or after introduction of the sperm into the oocyte. In one embodiment, the NAD⁺ agonist is introduced into the oocyte simultaneously with the sperm. Typically, the NAD⁺ agonist is introduced into the oocyte with the sperm. In one embodiment, the NAD⁺ agonist is introduced into the oocyte by injection, such as microinjection.

During IVF, oocytes are fertilized in vitro, and matured into blastocysts prior to transferring into a female. Blastocysts at a later stage of maturity are recognized as having a better chance at implanting and delivering a viable pregnancy. Not all zygotes fully develop into blastocysts, and the rate of blastocyst formation declines in oocytes from women of increasing reproductive age. It would be advantageous to provide a method of improving oocyte quality to improve blastocyst formation in vitro prior to implantation.

As described in the Examples, the inventors have found that oocytes harvested from mice treated with the NAD⁺ precursor NMN exhibit an enhanced ability to form blastocysts following fertilisation in vitro when compared to oocytes from mice not treated with NMN.

One aspect provides a method of improving or enhancing the ability of an oocyte to form a blastocyst during in vitro fertilisation, comprising introducing into the oocyte an agent which elevates SIRT2 activity or SIRT2 expression in the oocyte. In one embodiment, the agent is an NAD+ agonist. In one embodiment, the agent is an NAD+ precursor.

Another aspect provides an agent which elevates SIRT2 activity or SIRT2 expression in an oocyte for use in improving or enhancing the ability of an oocyte to form a blastocyst during in vitro fertilization; or use of an agent which elevates SIRT2 activity or SIRT2 expression in an oocyte in the manufacture of a medicament for improving or enhancing the ability of an oocyte to form a blastocyst during in vitro fertilization. In one embodiment, the agent is an NAD+ agonist. In one embodiment, the agent is an NAD+ precursor.

An oocyte has an enhanced ability to form a blastocyst if it has an increased probability of forming a blastocyst that can progress to pregnancy relative to that of an oocyte into which the agent has not been introduced.

In one embodiment, the agent (e.g., NAD⁺ agonist, NAD⁺ precursor) is introduced into the oocyte while the oocyte is in the female subject by administering to the subject an effective amount of the agent prior to obtaining the oocyte from the female subject for in vitro fertilisation.

In another embodiment, the agent is introduced into the oocyte in vitro. The agent may be introduced into the oocyte in vitro prior to and/or during fertilization of the oocyte. For example, if the agent is able to cross the cell membrane, the agent may be contacted with the oocyte by, for example, incubating the oocyte in medium containing the agent, for example, during in vitro maturation (IVM) from follicles, or during IVF. In some embodiments, the agent may be transfected or injected into the oocyte, for example, during intracytoplasmic sperm injection (ICSI).

As described in the Examples, the inventors have further found that there is a time dependent increase in efficacy up to 4 weeks of administration of NMN. Further, as described herein, the inventors have found that extended dosing of NMN through administering drinking water comprising NMN throughout the day has greater efficacy than administering a single daily dose of NMN by oral gavage.

Accordingly, in some embodiments, administration of the agent to the subject is carried out orally over a period of 1 or more weeks, typically 2 or more weeks, more typically 3 or more weeks, still more typically 4 or more weeks, for example, 2 to 8 weeks, more typically 3 to 7 weeks, still more typically about 4 to 6 weeks, prior to mating, or prior to obtaining oocytes from the subject. In one embodiments, the agent is administered at a dose of at least once per day. Typically, the agent is administered in multiple dosings per day (e.g., 2, 3, 4, 5, 6, 7, 8, 9, 10, 11, 12, 13, 14, 15, 16, 17, 18, 19 or 20 or more time per day). Typically, the agent is administered at multiple regular intervals per day. In some embodiments, the agent is administered by adding to a drinkable liquid (e.g. drinking water), or food, that is consumed regularly throughout the day.

In some embodiments, the agent is administered in a slow release format. Typically, the agent is administered once daily in a slow release format.

Also provided is an article of manufacture and a kit, comprising a container comprising an NAD⁺ agonist. In some embodiments, the container may be a bottle comprising the NAD⁺ agonist in oral dosage form, each dosage form comprising a unit dose of the NAD⁺ agonist. For example, apigenin in an amount for instance from about 100 mg to 750 mg, or NMN in an amount from about 100 mg to 750 mg.

In another embodiment, the container may be a bottle comprising the NAD⁺ agonist in injectable dosage form for use in ICSI.

The kit will further comprise printed instructions. The article of manufacture will comprise a label or the like, indicating treatment of a subject according to the present method.

All publications mentioned in this specification are herein incorporated by reference. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

In order to exemplify the nature of the present invention such that it may be more clearly understood, the following non-limiting examples are provided.

### EXAMPLES

Experimental Procedures

Animals

Animals: All mice were on the C57BL6J/Ausb genetic background. Mice were maintained on a 12 hr light cycle (0700/1900) in individually ventilated cages at 22±1° C., 80% humidity at a density of 5 mice per cage. Animals were fed a standard chow diet from Gordon's Specialty Feeds (Yanderra, NSW Australia) comprising 8% calories from fat, 21% calories from protein, and 71% calories from carbohydrates, with a total energy density of 2.6 kcal/g. Alternatively, animals were fed a high fat diet (HFD) as indicated, which was 45% calories from fat (beef lard), 20% calories from protein, and 35% calories from carbohydrate at a density of 4.7 kcal/g, based on rodent diet D12451 (Research Diets, New Brunswick, N.J.). Animals were used at ages as indicated in figures. Transgenic mice expressing a SIRT2 trangene (SIRT2-Tg), NMNAt3 transgene (NMNAT3-Tg) and NMNAT1 transgene (NMNAT1-Tg) mice were virgin females, and comparisons were made between transgenic animals and their wild-type littermates.

Oocyte Collection

To assess oocyte yield, and mice were hormonally stimulated to superovulated by intraperitoneal injection with 7.5 IU/mL of Pregnant Mare's Serum Gonadotropin (PMSG) (Folligon; Intervet, Boxmeer, Holland) to stimulate follicular growth. After 44-46 hours, ovaries were collected in HEPES-buffered minimum essential medium (aMEM; Gibco Life Technologies, Grand Island, N.Y.) supplemented with 50 82 M of 3-isobuty-1-methylxanthine (IBMX) (Sigma aldrich, NSW, AU) (M2 medium) to maintain meitotic arrest at germinal vesicle stage. Cumulus oocyte complexes (COCs) were isolated from preovulatory follicles using a 27-gauge needle and collected using flame-pulled borosilicate Pasteur pipettes in M2 medium supplemented with 3 mg/ml bovine serum albumin (BSA; Sigma Aldrich, St. Louis, Mo.) and 100 μM IBMX (Sigma Aldrich). Cumulus oocyte complexes (COCs) were mechanically denuded of cumulus cells by pipetting, and the denuded oocytes were transferred to another dish containing a drop of fresh M2-medium plus IBMX on heat block with 37° C. with lid to protect oocytes from light.

Western Blotting

SDS-PAGE and Western blot analysis were performed according to standard procedures and detected with the ECL detection kit (Bio-rad, Australia). For Western blot analysis antibodies directed against SIRT2 (Sigma), BubR1 (Novus), and Tubulin (Sigma), were used.

Histology

Ovaries were dissected from freshly euthanased animals, and preserved in 10% neutral buffered formalin for 24 hr, followed by 70% ethanol, until embedding in paraffin blocks. Blocks were sectioned on a microtome and subjected to haematoxylin and eosin (H&E) staining. Primordial follicles were manually counted by a blinded investigator.

Statistical Analysis

Data are presented as means±standard deviations. Statistical significance was performed using twotailed Student's t test or two-way ANOVA test with post-hoc Tukey test. Statistical test was performed using GraphPad Prism software. P values of less than 0.05 were considered statistically significant.

Results

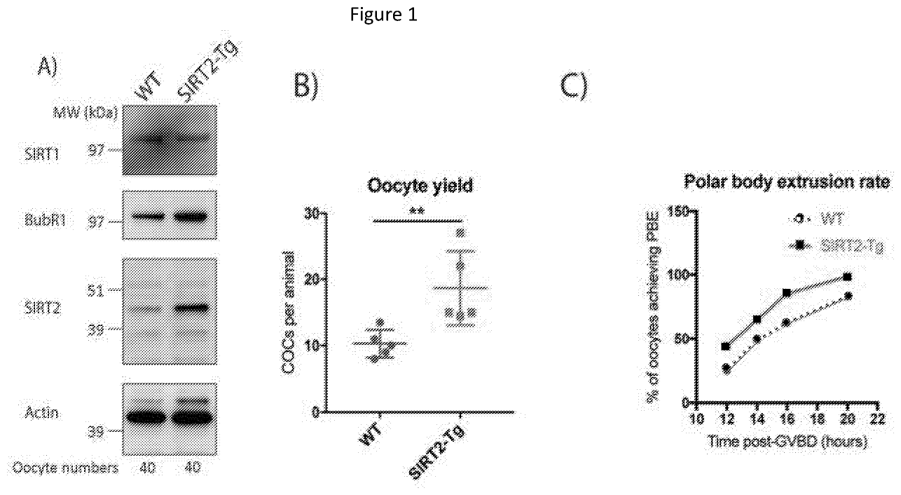

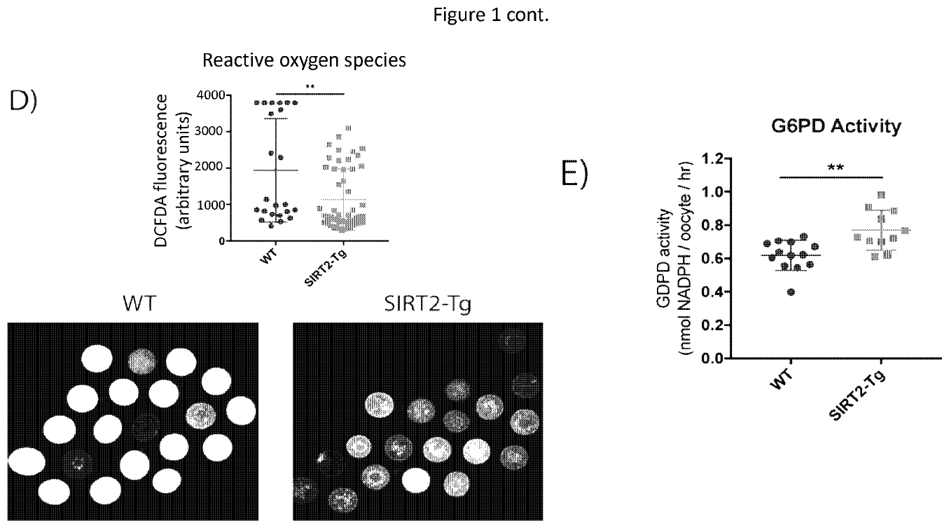

BubR1 is susceptible to ubiquitination and degradation following acetylation at a key residue, Lys668. Deacetylation of this site by the NAD⁺ dependent deacetylase SIRT2 stabilises BubR1 levels, and we hypothesised that increased SIRT2 levels might preserve BubR1 levels in oocytes, and have improved fertility. To investigate this, we obtained a previously described strain of mice which globally over-express SIRT2 (North et al. (2013) EMBO J. 33: 1438-1453). Consistent with our hypothesis, oocytes from SIRT2-Tg animals had higher levels of BubR1 (FIG. 1A) compared to their wild-type (WT) littermates. To investigate whether this translated into an improved ovarian reserve, animals were hormonally primed to induce ovulation, and oocyte release was measured. In 3 month-old animals, SIRT2 over-expression resulted in a 2-fold increased yield in cumulus oocyte complexes (COCs), demonstrating a role for SIRT2 in maintaining ovarian reserve (FIG. 1B). These oocytes were then matured in vitro, and meiotic progression was assessed through the proportion of oocytes undergoing polar body extrusion (PBE), a key event in the second stage of meiosis (FIG. 1C). As shown in FIG. 1C, at all timepoints, SIRT2-Tg oocytes demonstrated consistently higher PBE rates, demonstrating that oocytes were of an improved quality.

In addition to its role in stabilising BubR1, SIRT2 also deacetylates and maintains the activity of the pentose phosphate enzyme glucose-6-phosphate dehydrogenase (G6PD), which regenerates levels of the cellular antioxidant glutathione, through its role as the primary source of NADPH in the cell, needed to regenerate oxidised glutathione (GSSG) into reduced glutathione (GSH). Oxidative stress due to GSH insufficiency is also thought to be a key contributing factor in oocyte dysfunction, and to assess whether SIRT2 plays a role in preventing this, oocytes from aged, 12 month-old SIRT2-Tg and WT littermates were subjected to oxidative stress through H₂O₂ exposure. ROS levels were then assessed in individual oocytes using the ROS sensitive fluorescent stain DCFDA, and confocal microscopy. Consistent with the role of SIRT2 in increasing GSH, oocytes from SIRT2-Tg animals had increased staining for DCFDA (FIG. 1D). To assess whether the increase in GSH levels was indeed due to improved G6PD activity, this enzyme was assayed directly, and found to be increased in oocytes from SIRT2-Tg animals (FIG. 1E). These data support the hypothesis that SIRT2 suppresses ROS during ageing in oocytes through maintaining the activity of the enzyme G6PD.

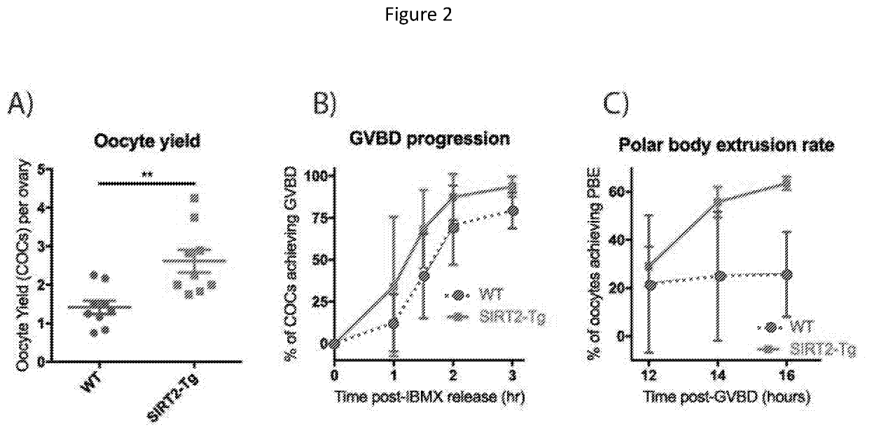

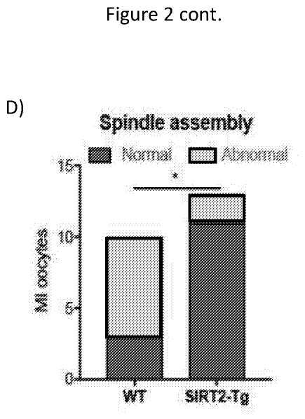

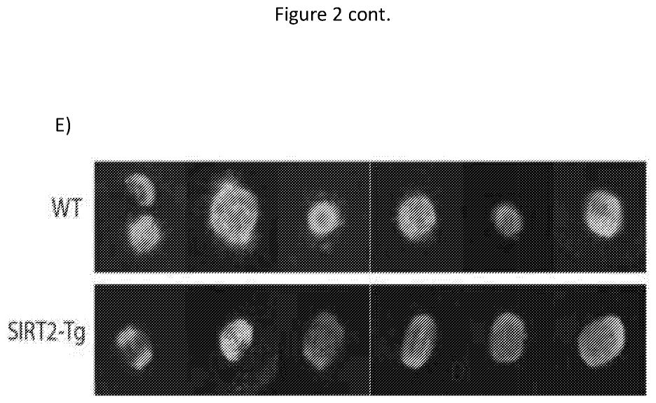

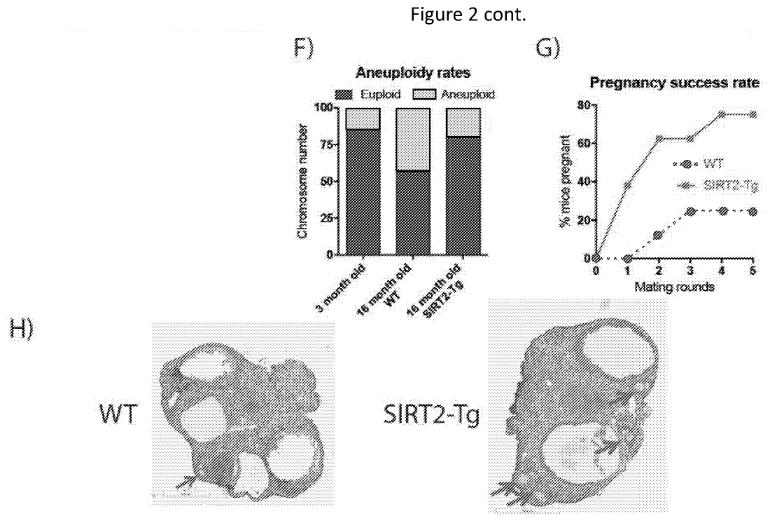

Next, we aimed to determine whether SIRT2 played a role in maintaining fertility during ageing. SIRT2-Tg and WT animals were aged to 12 months, an age at which mice are typically infertile. Animals were hormonally primed, and COCs were collected, followed by in vitro maturation. As in young mice, SIRT2-Tg animals again yielded more than twice the number of oocytes as their WT littermates (FIG. 2A). Strikingly, only 25% of oocytes collected from aged WT animals could complete PBE, compared to over 60% of oocytes from SIRT2-Tg littermates (FIG. 2C). Given that BubR1 plays a key role in kinetochore attachment to spindles during meiosis, we next sought to determine whether this change in meiotic progression was due to alterations in spindle structure. Confocal microscopy of immunostained oocytes showed that aged WT oocytes showed strikingly disordered spindle arrangements, with chromsomes poorly aligned (FIG. 2D). In contrast, oocytes from SIRT2-Tg oocytes maintained the classic barrel-shaped, bipolar spindle structure, with chromosomes clearly aligned. Together, these data point towards an essential role for SIRT2 in maintaining ovarian reserve and oocyte quality. SIRT2 is a lesser studied member of the sirtuin family, when compared to SIRT1, which plays a highly prominent role in biological ageing. To test whether the improvements in oocyte yield and spindle assembly were common to other sirtuins, we also obtained a strain of mice which globally over-expressed SIRT1, and observed no change in these parameters (FIG. 2E), suggesting that these effects may be unique to SIRT2 and are not shared by other members of this family.

We next tested whether oocytes from aged SIRT2-Tg animals were less prone to aneuploidy, a key feature of oocyte dysfunction with advancing maternal age. Oocytes were harvested from superovulated animals, and treated with monastrol to allow for chromosome number to be assessed in situ by confocal microscopy. While the rate of aneuploidy (less than or greater than 20 chromosomes per oocyte) was 15% in oocytes from 2 month-old animals, and 43% in WT versus 20% in SIRT2-Tg oocytes from 14 month-old animals, this trend did not reach statistical difference (FIG. 2F).

To determine whether this translated to improvements in fertility, a separate cohort of animals was aged to 16 months, well past the typical age of infertility in this species, and subjected to repeated rounds of mating with stud males of proven fertility. Successful mating was confirmed by the presence of vaginal plugs, and pregnancies were determined two weeks later by micro-ultrasound imaging for the presence of foetal heartbeat(s). Starting at 16 months of age, only 25% of WT females achieved pregnancy over 5 mating rounds, while this was tripled to 75% of SIRT2-Tg females (FIG. 2G). Together, these data demonstrate that SIRT2 plays an essential role in maintaining fertility during ageing.

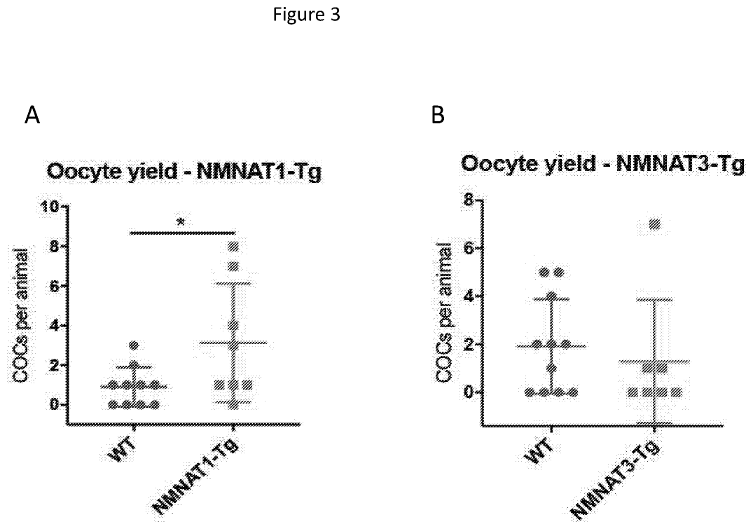

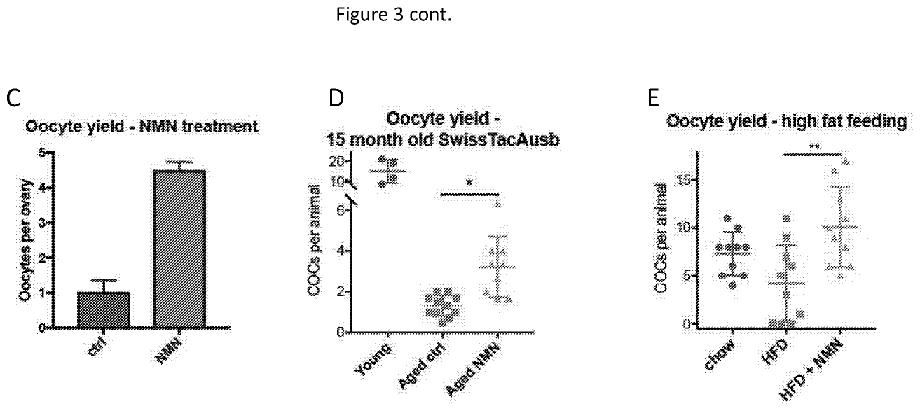

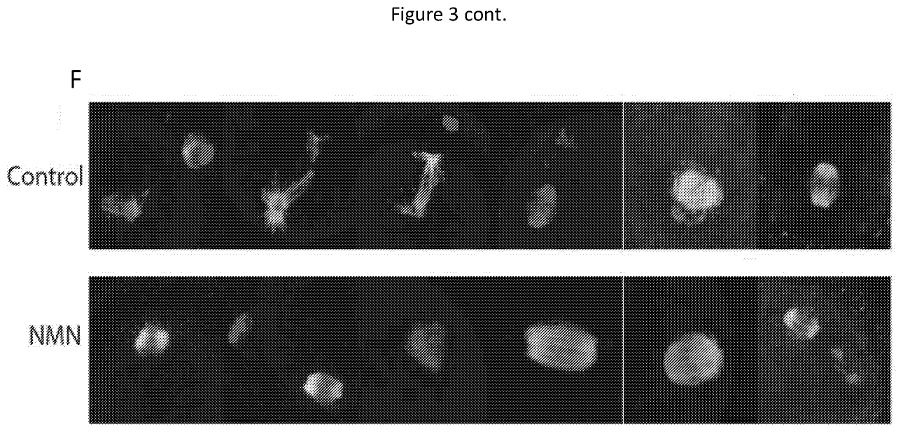

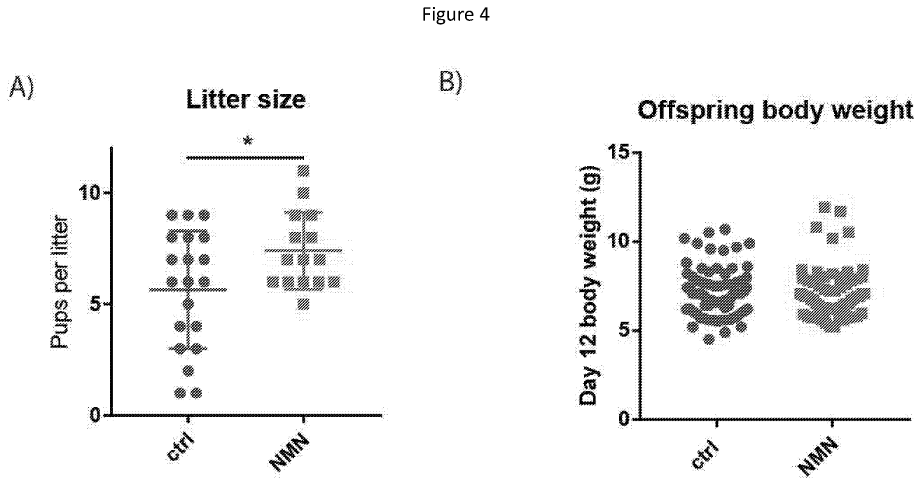

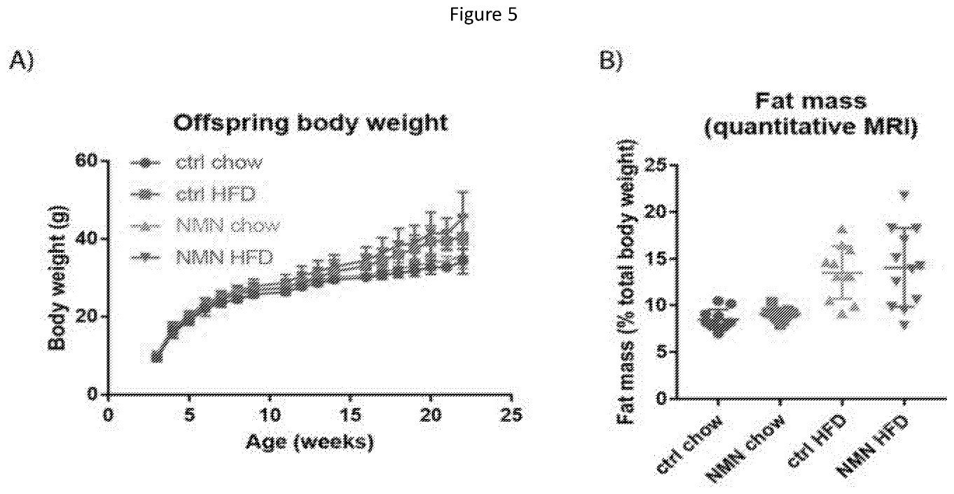

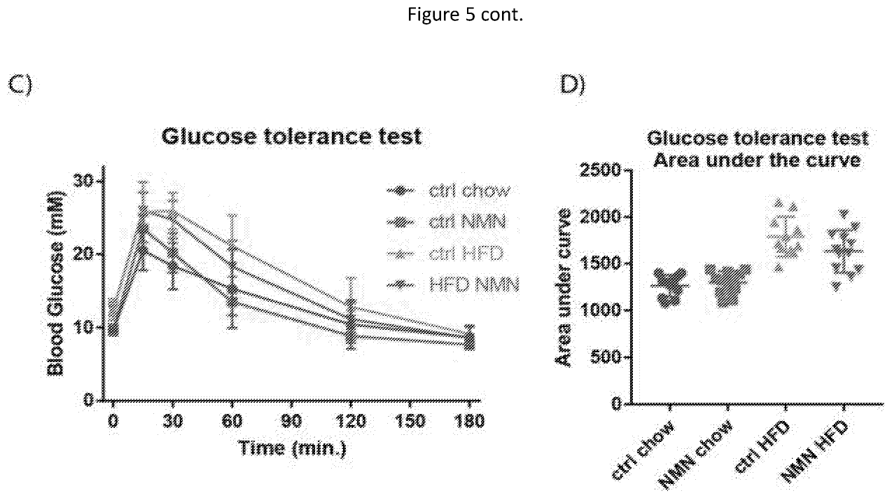

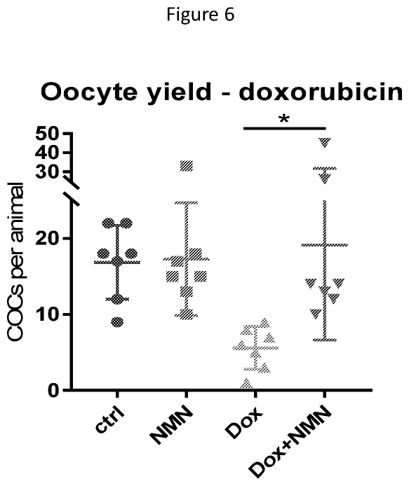

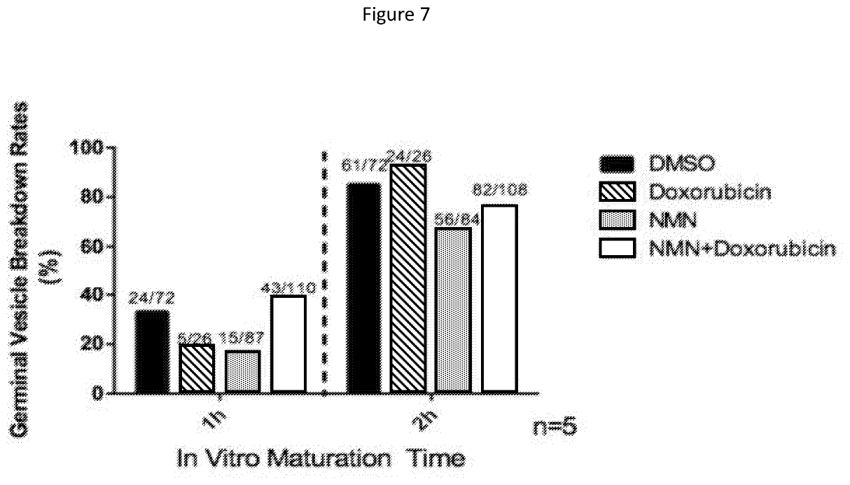

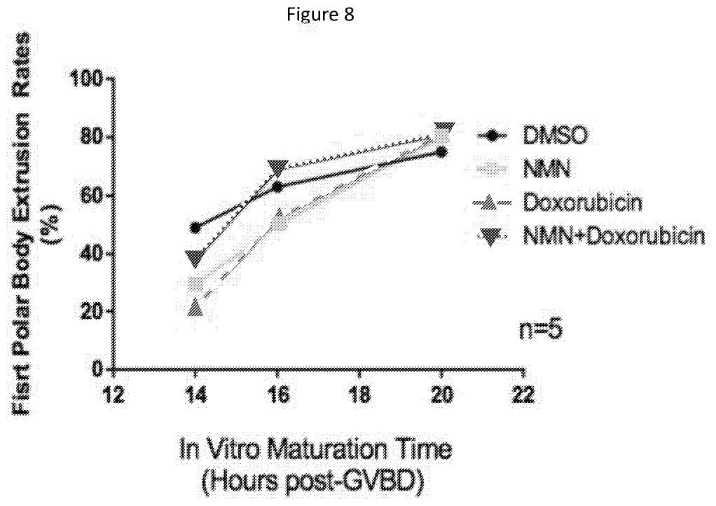

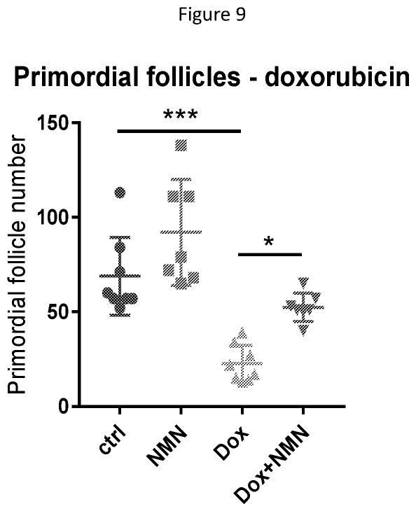

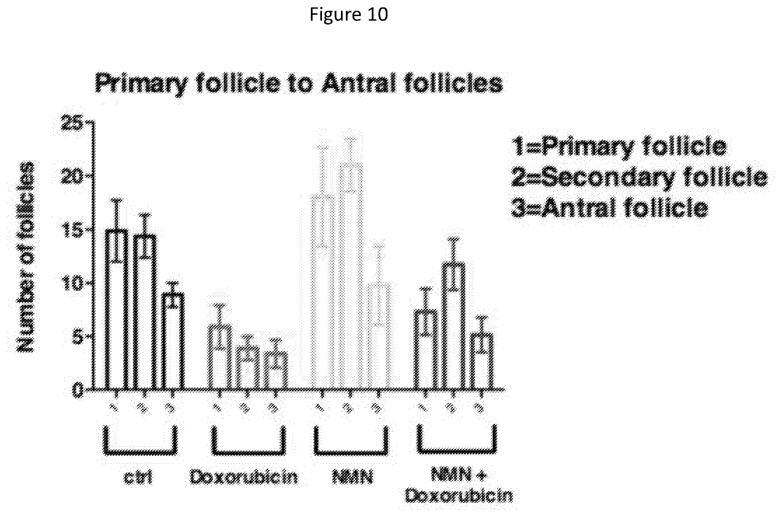

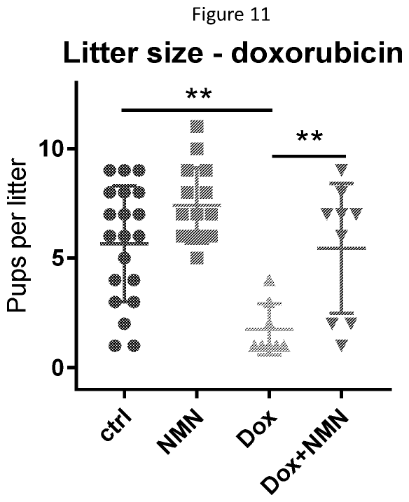

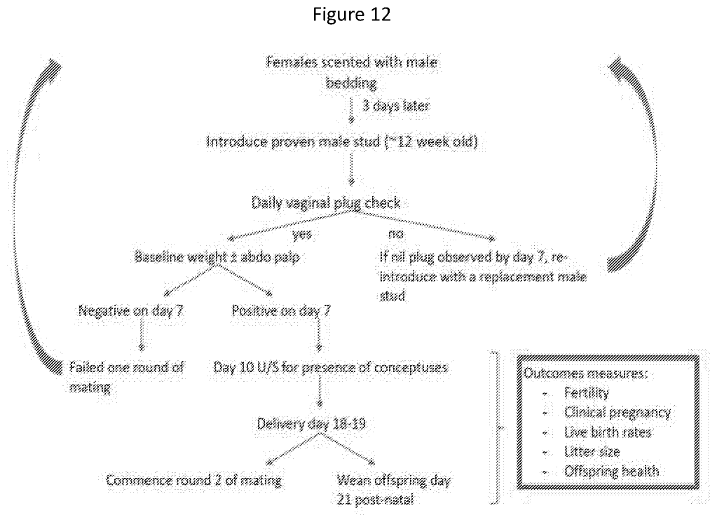

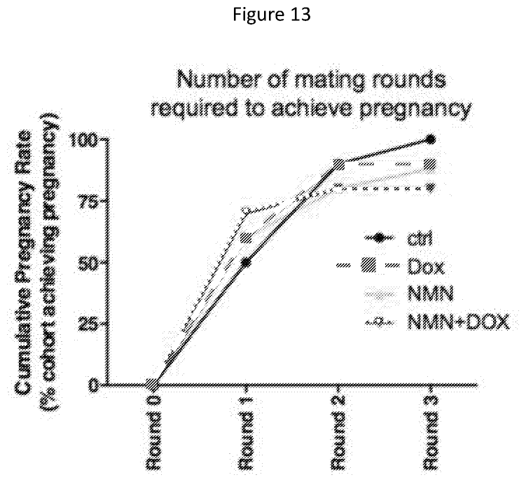

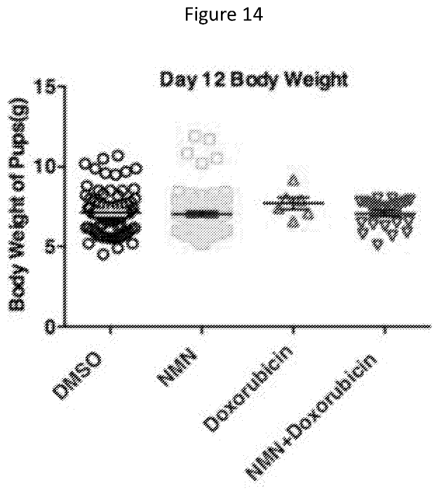

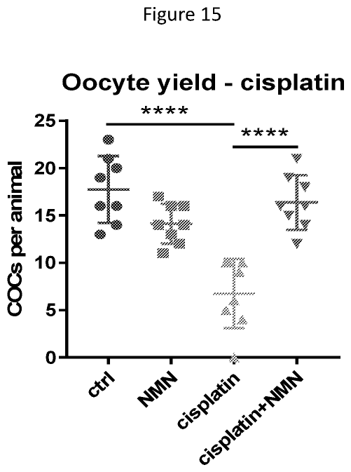

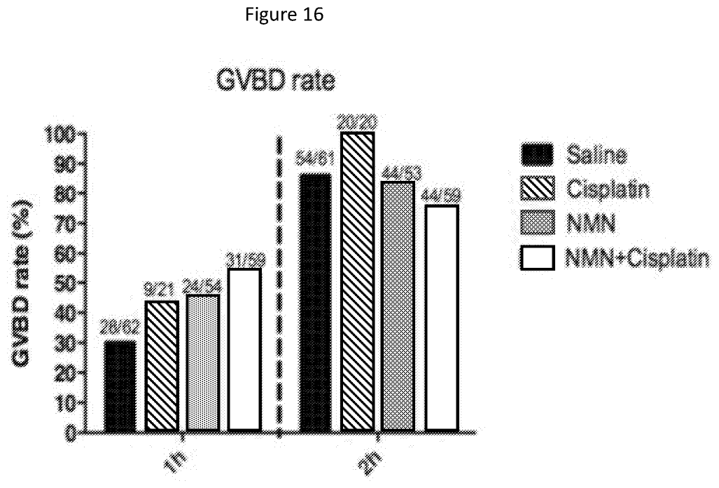

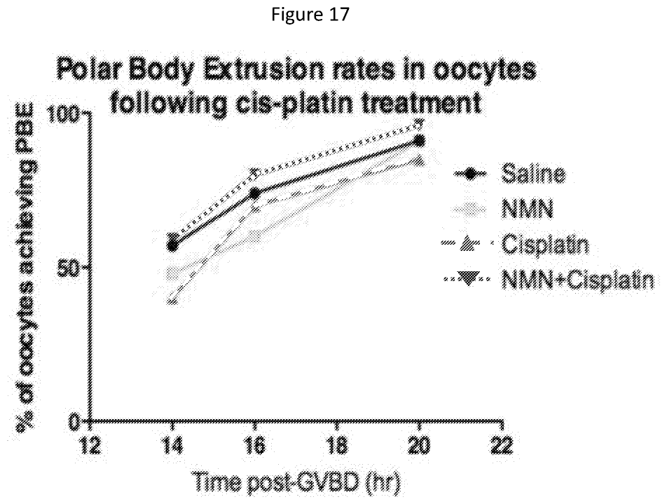

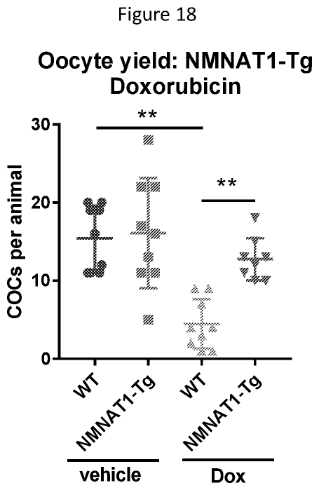

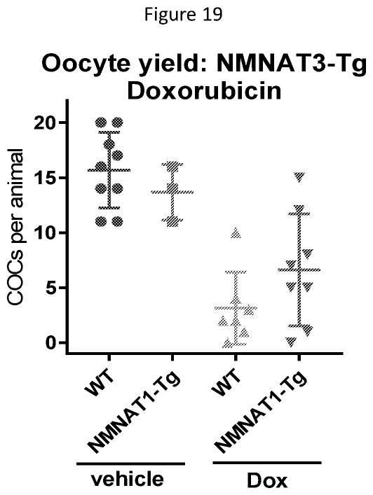

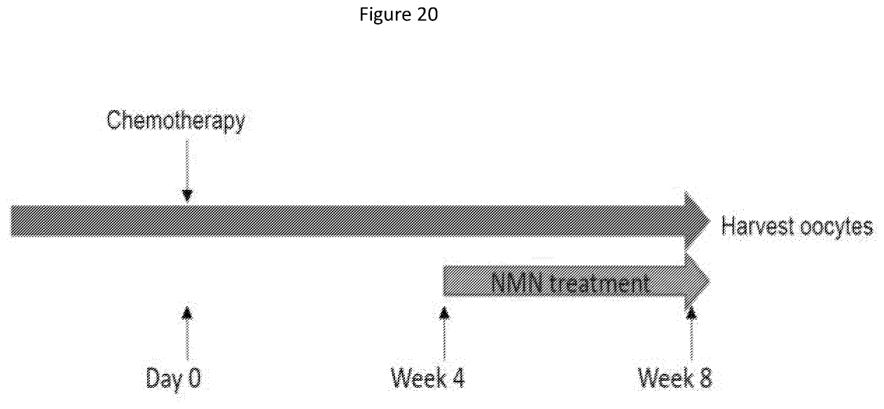

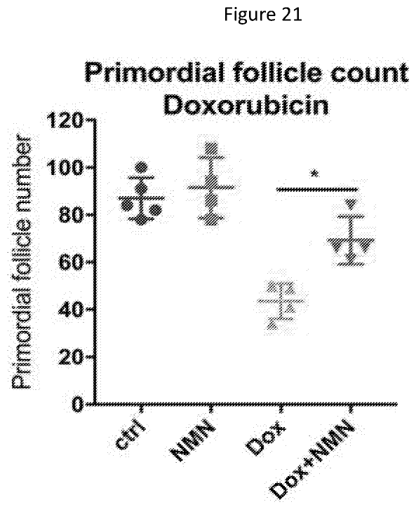

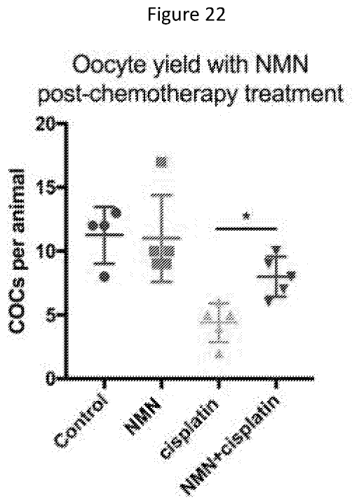

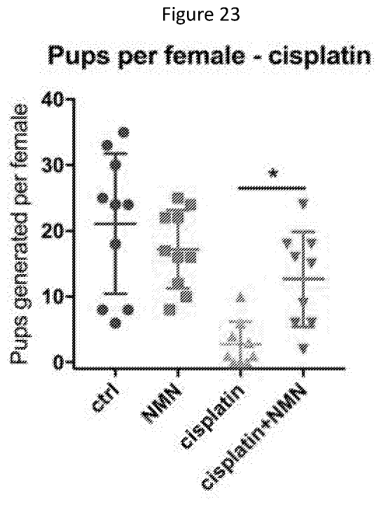

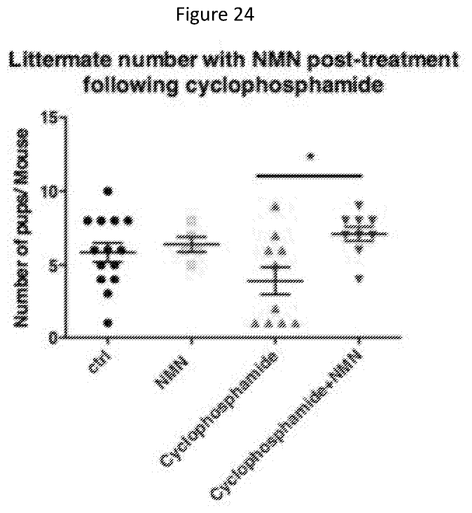

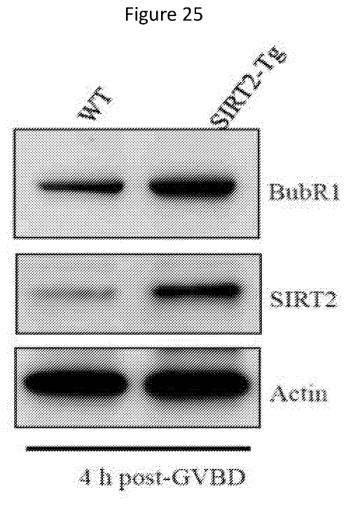

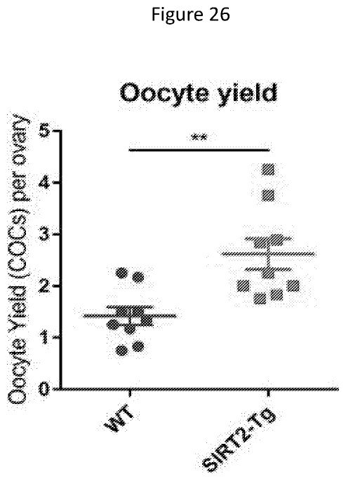

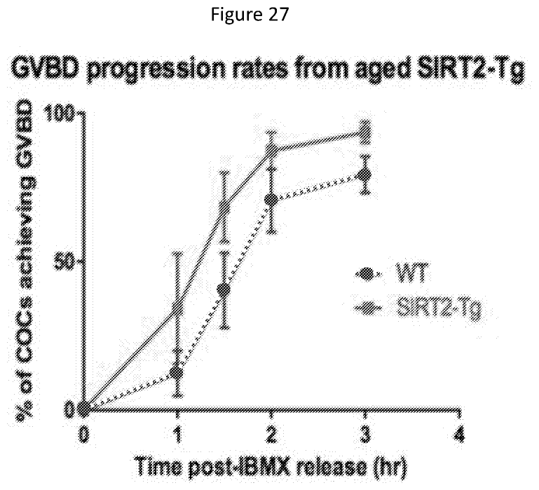

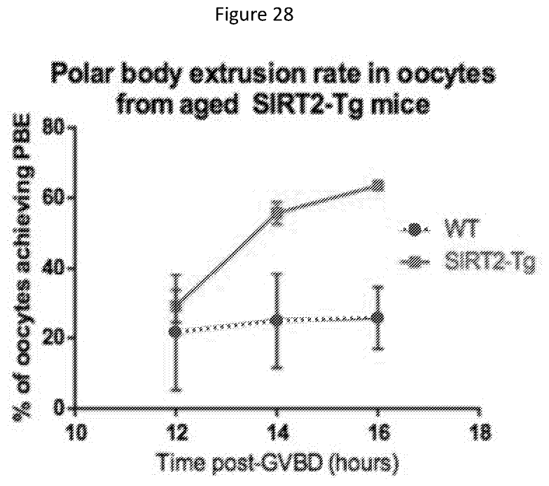

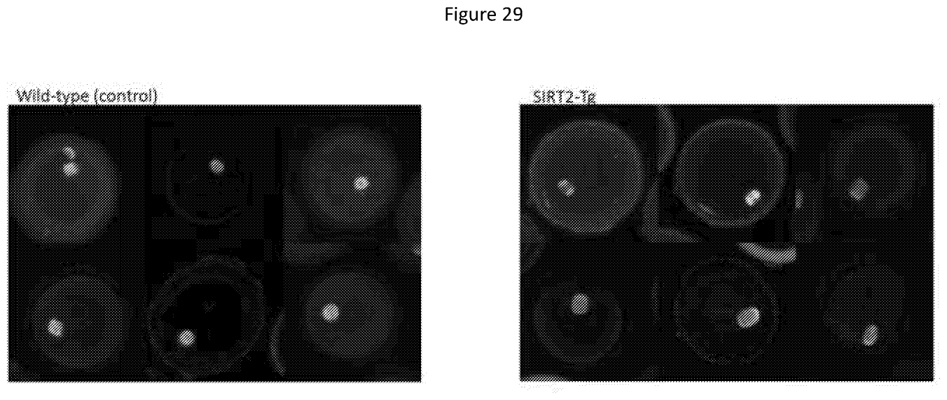

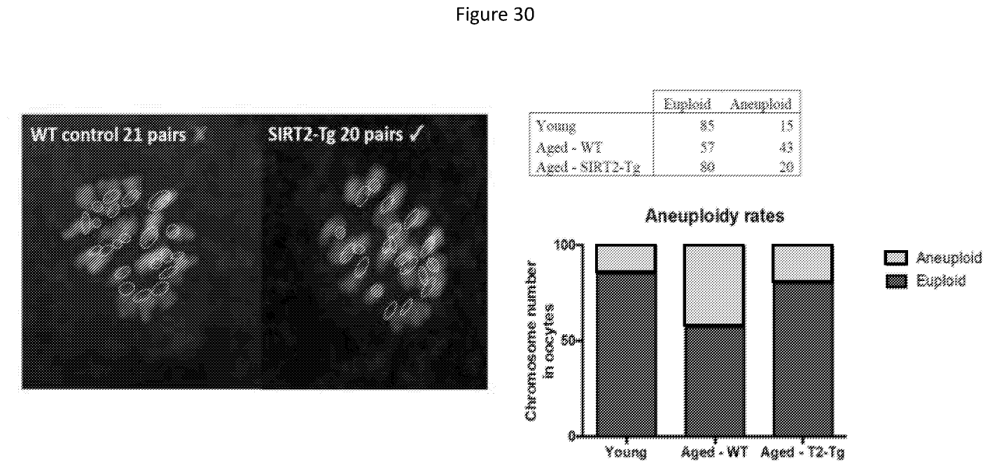

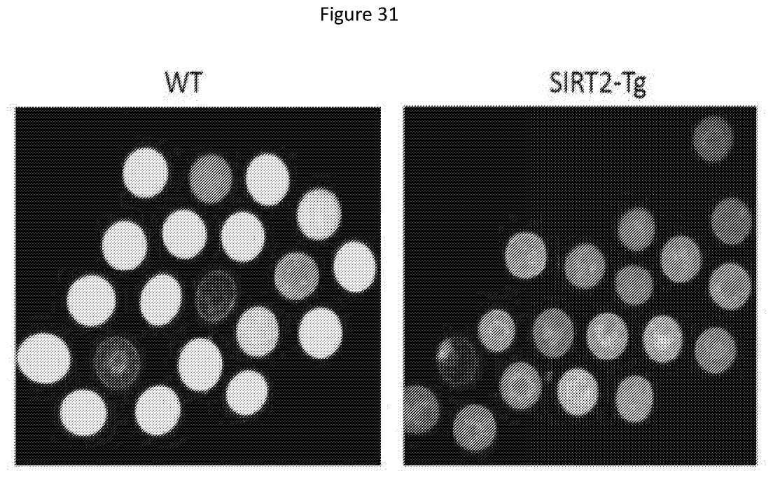

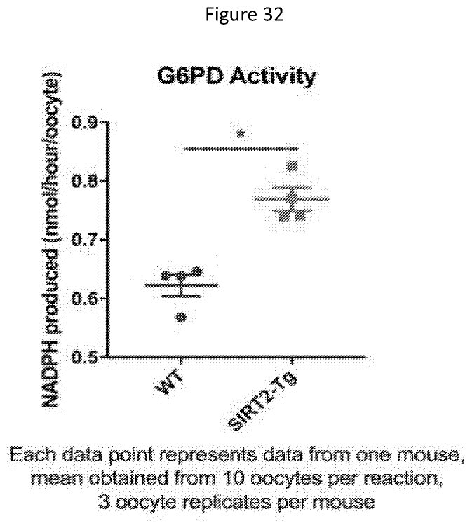

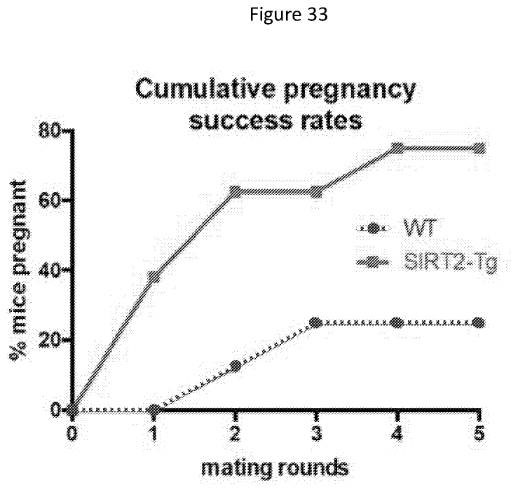

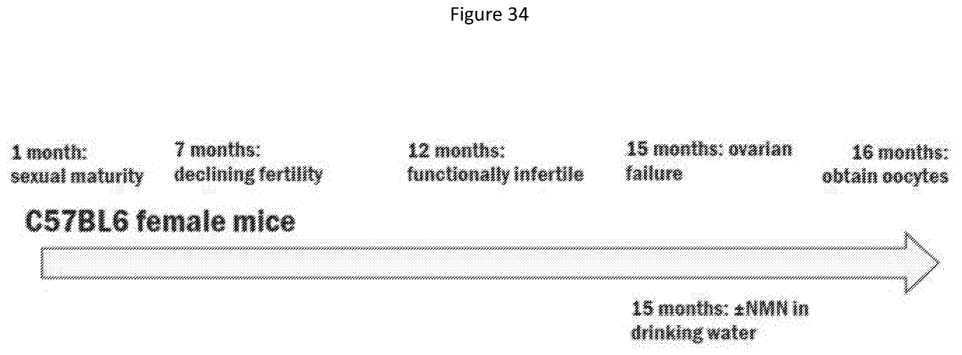

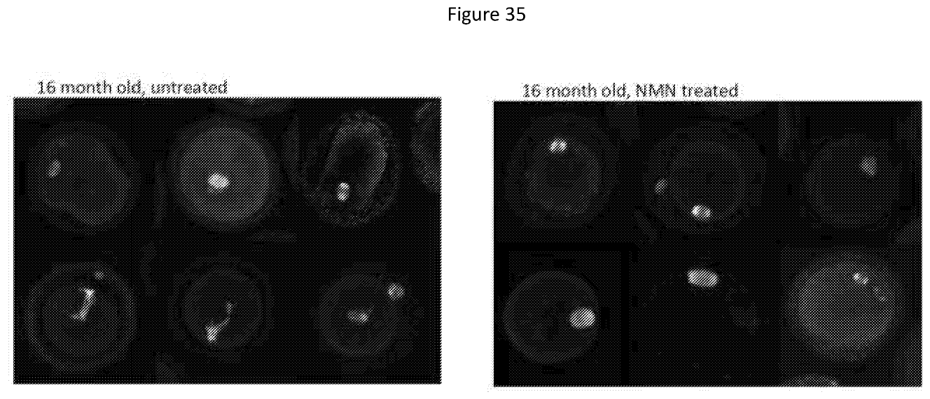

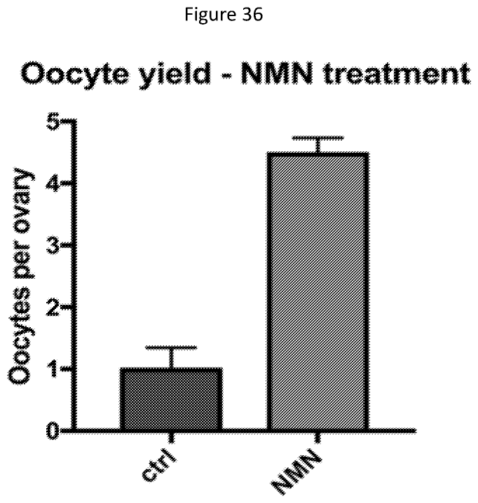

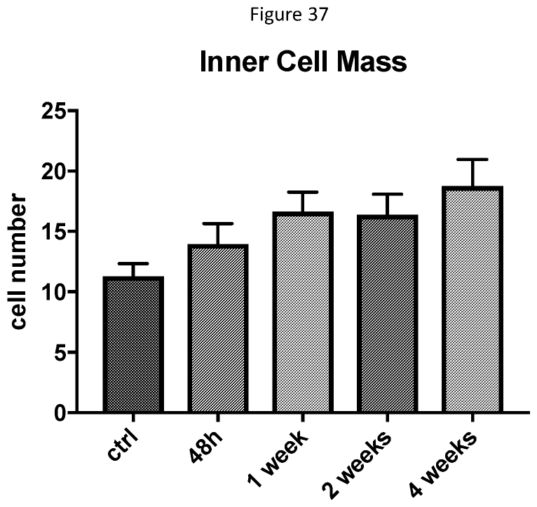

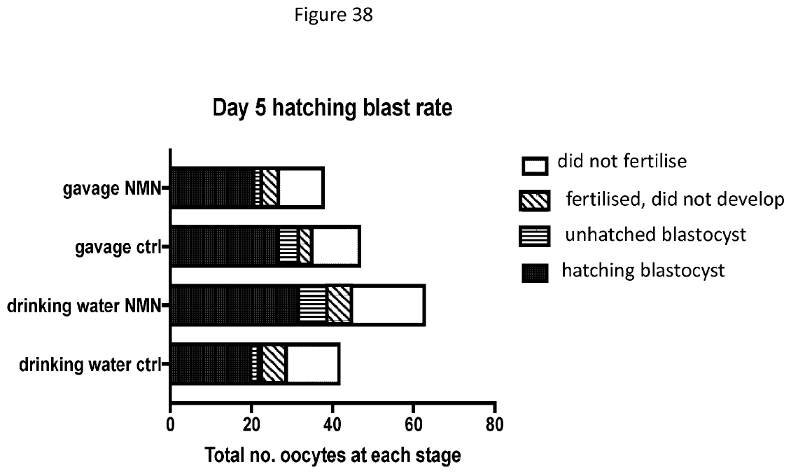

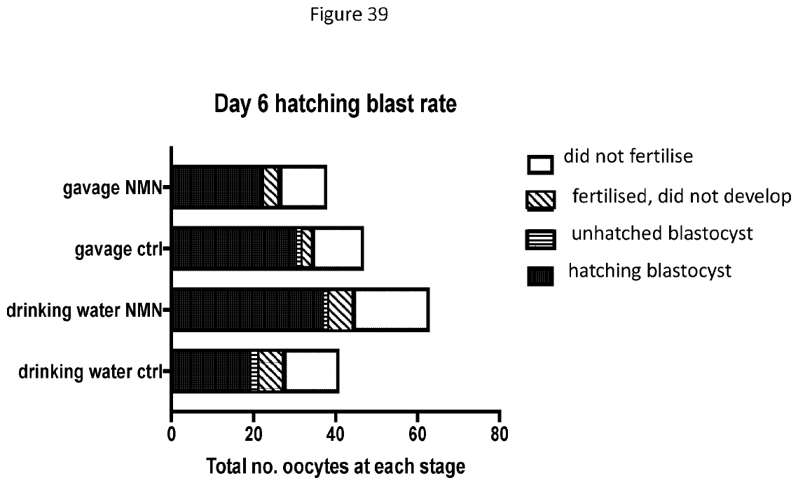

As with other members of the sirtuin family, SIRT2 is critically dependent upon the availability of nicotinamide adenine dinucleotide (NAD⁺), a cofactor that is consumed during the reaction it carries out. NAD⁺ levels decline during biological ageing, impairing the ability of sirtuins to carry out their reaction. We hypothesised that the reason for declining SIRT2 activity during old age might be a decline in NAD⁺ levels in oocytes. As hypothesised, we observed a steady decline in NAD⁺ levels in oocytes from animals with advancing age. NAD⁺ is either synthesised de novo from tryptophan, in the Preiss-Handler pathway, or recycled via the NAD salvage pathway. Both pathways require nicotinamide mononucleotide adenylyl transferase activity, catalysed by the three members of the NMNAT enzyme family (NMNAT1-3), as either the last or second-last step in NAD⁺ synthesis. The members of this family exhibit different subcellular localisations, with NMNAT1 present in the nucleus, and NMNAT3 present in the mitochondria. We obtained two transgenic strains of mice which globally over-express the enzymes NMNAT1 (NMNAT1-Tg mice) and NMNAT3 (NMNAT3-Tg mice) (described in Yahata N et al J. Neurosci. 29 (19) 6276-6284 2009), and maintained females until the age of 14 months, past the normal period of fertility for this species. As with oocytes from SIRT2-Tg mice, NMNAT1-Tg animals displayed an increased yield in COCs following hormonal super-ovulation (FIG. 3A), supporting the idea that NAD⁺ synthesis might support SIRT2 activity and oocyte competence. Interestingly, aged NMNAT3-Tg mice did not exhibit any change in oocyte number (FIG. 3B), suggesting that NAD⁺ levels in the nuclear compartment, and not the mitochondrial compartment, is more important to oocyte development. It is also worth noting that this nuclear compartment of NAD⁺ is likely to merge with the cytosolic compartment during the nuclear envelope breakdown that occurs in oocytes.

Systemic treatment with the NAD⁺ precursor nicotinamide mononucleotide (NMN) increases intracellular NAD⁺ levels, and we hypothesised that NMN treatment could restore the activity of SIRT2 to stabilise BubR1 and maintain oocyte reserve and competence into later ages. We obtained 15 month-old female mice, an age at which this species is functionally infertile, and treated them with NMN through addition to drinking water (2 g/L) for 4 weeks. Animals were super-ovulated, and oocyte yield was assessed. In two genetically distinct strains of mice, C57BL6 and SwissTacAusb, COC yield in aged females was more than doubled following NMN treatment (FIG. 3C, 3D). Obesity is a major risk factor for female infertility, and to assess whether a similar mechanism was at work, we subjected young SwissTacAusb female mice to 3 months of high fat feeding, followed by NMN treatment for 4 weeks. As in aged mice, NMN treated high fat fed mice delivered a higher oocyte yield than their untreated, high fat fed littermate controls (FIG. 3E), providing a third model to support the ability of NMN treatment to enhance female fertility.

To determine the impact of NMN on spindle assembly, oocytes from C57BL6 mice were next stained for spindle structure (FIG. 3F). Strikingly, oocytes from aged, untreated animals showed highly disordered spindle and chromosome arrangements, whilst oocytes from NMN treated littermates displayed a bi-polar, barrel-shaped arrangement with well-aligned chromosomes (FIG. 3F). These data are consistent with the hypothesis that pharmacological restoration of NAD⁺ can reverse the age-dependent decline in BubR1 levels, which is needed to restore kinetochore attachment to spindles. Histological analysis of ovaries from NMN treated females showed an improved oogonial reserve, which may be a result of increased BubR1 levels and an improved ability to retain oocytes in prophase I.

To determine whether NMN treatment could improve overall, functional fertility, we conducted a breeding trial in C57BL6 mice. Both pregnancy success rates and litter size (FIG. 4A) were improved in animals that received NMN, further confirming the importance of this mechanism to female fertility. Offspring from females that received NMN treatment developed at a similar rate to offspring from untreated females, even after challenging with high fat feeding (FIG. 5A and 5B). To ascertain whether there were differences in metabolic homeostasis, animals were subjected to glucose tolerance tests (FIG. 5C and 5D), with no change between offspring from NMN treated and untreated females, suggesting that NMN does not adversely affect development in offspring.

In humans, the oocyte is the primary determinant of female fertility during ageing. Patients undergoing IVF with their own oocytes display an age dependent decline in pregnancy success rates, while IVF patients using donor oocytes display a constant pregnancy success rate, regardless of maternal age, highlighting the importance of oocytes over other elements of the reproductive milieu in maintaining fertility during ageing. Unfortunately, there is as yet no therapy capable of improving oocyte quality. Gonadotrophin therapy promotes the maturation of follicles in the ovary, to improve oocyte release, but does not alter oocyte competence. IVF is the dominant form of ART, with a low pregnancy success rate, which is severely constrained by oocyte quality.

Here, we show for the first time that systemic NAD⁺ availability is a primary determinant of oocyte quality during biological ageing in mammals, through a mechanism that involves SIRT2, BubR1, and the maintenance of spindle attachment to kinetochores during meiosis. We provide data from two genetic models, and pharmacological data from three different models of challenged female fertility, to support these claims. This molecular mechanism offers the opportunity of a clinically tractable pathway for the treatment of female infertility. Given the worldwide trend for delayed age of maternity, there is an increasing incidence of offspring born with chromosomal disorders, such as Trisomy 21. The approach shown here may offer the widespread opportunity to maintain fertility and lower the chance of chromosomal disorders in offspring. Finally, given the low success rates of ART, the high incidence of hospitalisation for women undergoing ART from hormonal stimulation, and the high rate of unintended and clinically risky multiple pregnancies, these findings are of critical importance to improving future outcomes in ART.

### Example 2

The aim of this investigation was to determine whether treatment with the NAD+ raising compound nicotinamide mononucleotide (NMN) could protect against chemotherapy induced infertility, using the anthracycline chemotherapy drug doxorubicin, a commonly used mainstay of modern chemotherapy.

8 week-old C57BL6 female mice were treated with a single dose of either doxorubicin (10 mg/kg) or a vehicle control through i.p. injection in a 100 uL volume, in the presence or absence of co-treatment with the NAD⁺ precursor nicotinamide mononucleotide (NMN). NMN was delivered through addition to drinking water at 2 g/L, for a final dose of approximately 165 mg/kg. Administration of NMN began one day prior to doxorubicin administration. At 2 months post doxorubicin, animals were superovulated using PMSG treatment (i.p. injection), and 42 hr later, animals were euthanized, and ovaries were dissected. Ovaries were then punctured to release MI oocytes. In a separate cohort of animals, the same experiment was repeated using animals treated with the platinum based chemotherapy drug cisplatin. In FIG. 6 and Table 1, numbers of cumulus oocyte complexes are listed.

TABLE 1
Numbers of cumulus oocyte complexes released from mice
treated with or without doxorubicin, in the presence or absence
of NMN. Data were analysed by 2-way ANOVA with a post-hoc
Tukey test.

| | | | Dox + |
|---|---|---|---|
| Ctrl | NMN | Dox | NMN |
| 12 | 13 | 5 | 12 |
| 9 | 33 | 1 | 26 |
| 18 | 17 | 8 | 45 |
| 18 | 15 | 9 | 14 |
| 17 | 10 | 3 | 14 |
| 22 | 15 | 6 | 10 |
| 22 | 18 | 7 | 13 |

There are two types of oocytes, cumulus oocyte complexes (COCs), and denuded oocytes, with COCs being covered in a layer of protective somatic cells, and of generally higher quality, typically used in IVF. These data suggest that NMN treatment is able to protect against doxorubicin induced oocyte loss.

Once harvested from ovaries, oocytes were stored in IBMX media to prevent progression into meiosis. Once released from IBMX, meiotic progression was assessed by the proportions of oocytes achieving germinal vesicle breakdown (GVBD) for meiosis I, and polar body extrusion (PBE) for meiosis II, with results shown in FIGS. 7 and 8 and Tables 2 and 3. These data suggest that there is no defect in meiotic progression rates in oocytes which survive chemotherapy treatment.

TABLE 2
Proportions (numbers showing % of total) of harvested
oocytes achieving germinal vesicle breakdown at indicated
timepoints, following release from IBMX.

| | ctrl | Dox | NMN | DOX + NMN |
|---|---|---|---|---|
| 1 h | 33 | 19 | 17 | 39 |
| 2 h | 85 | 92 | 67 | 76 |

TABLE 3
Proportions of oocytes achieving polar body extrusion
at indicated timepoints, following GVBD

| | ctrl | NMN | Dox | NMN + Dox |
|---|---|---|---|---|
| 14 hr | 49 | 79 | 21 | 38 |
| 16 hr | 63 | 50 | 52 | 69 |
| 20 hr | 75 | 80 | 81 | 81 |

In a separate cohort of animals, doxorubicin and NMN treatment was carried out as before, and at 2 months of age, animals were euthanased in the absence of hormonal stimulation, and ovaries harvested for histological analysis. Ovaries were dissected and preserved in 10% neutral buffered formalin for 24 hr, following which they were moved to 70% ethanol until wax embedding, sectioning, and H&E staining. H&E sections were analysed in a blinded fashion to count primordial (FIG. 9) and later stage follicles (FIG. 10), which are indicative of ovarian reserve.

Following oocyte counting and analysis of oocyte meiotic progression, as well as a separate cohort of animals for histological ovarian analysis, a separate cohort of animals were treated as indicated, and mated with stud males of proven track record, to assess the ability of animals to deliver live births following chemotherapy treatment in the presence of absence of NMN co-treatment. These data show no change in the ability to achieve pregnancy (FIG. 13 and Table 4), however a strong reduction in the number of pups born per litter following doxorubicin treatment, rescued by NMN co-treatment (FIG. 11 and Table 5).

TABLE 4
Number of mating rounds required to achieve pregnancy

| | ctrl | NMN | Dox | Dox + NMN |
|---|---|---|---|---|
| Round 0 | 0 | 0 | 0 | 0 |
| Round 1 | 50 | 60 | 60 | 70 |
| Round 2 | 90 | 80 | 90 | 80 |
| Round 3 | 100 | 88 | 90 | 80 |

TABLE 5
Female C57BL6 mice treated with doxorubicin and/or NMN
were mated, and the number of live pups born per litter
recorded.

| ctrl | NMN | Dox | Dox + NMN |
|---|---|---|---|
| 4 | 6 | 4 | 2 |
| 3 | 11 | 1 | 7 |
| 9 | 7 | 1 | 1 |
| 1 | 7 | 1 | 2 |
| 6 | 6 | 1 | 8 |
| 7 | 9 | 3 | 9 |
| 8 | 5 | 1 | 7 |
| 9 | 6 | 2 | 6 |
| 8 | 9 | | 7 |
| 6 | 7 | | |
| 7 | 8 | | |
| 9 | 10 | | |
| 8 | 8 | | |
| 4 | 6 | | |
| 2 | 6 | | |
| 1 | | | |
| 6 | | | |
| 5 | | | |
| 7 | | | |
| 3 | | | |

Pups born to female mice receiving NMN show no difference in body weight (FIG. 14), providing evidence that this treatment is not toxic to offspring if provided during pregnancy.

The design for this mating trial experiment is shown in FIG. 12.

Together these data suggest that NMN can prevent loss of oocyte numbers following treatment with the anthracycline doxorubicin, and the platinum drug cisplatin, two widely used chemotherapeutic drugs. Further, these data show that at a functional level, NMN can prevent a loss in fertility, as determined by the number of pups born per litter, during doxorubicin treatment. These data indicate that NMN may be used as a method to prevent infertility in female patients undergoing chemotherapy treatment.

Example 3

### Protection Against Cisplatin Induced Infertility with NMN

The aim of this investigation was to determine whether treatment with the NAD⁺ raising compound nicotinamide mononucleotide (NMN) could protect against chemotherapy induced infertility, using the platinum-based chemotherapy drug cisplatin, one of the oldest and still most commonly used chemotherapy drugs since its discovery in the mid twentieth century. As with Experiment 1 above, seven week-old C57BL6 female mice received a single dose of cisplatin (5 mg/kg in saline, i.p. injection) in the presence or absence of NMN treatment, through addition to drinking water (2 g/L for a final dose of 155 mg/kg). Two months later, animals were hormonally stimulated with PMSG, and 42 hr later, euthanased, ovaries harvested, and then punctured to release oocytes. As observed in FIG. 15 and Table 6, cisplatin treatment reduces oocyte yield, which is completely rescued by NMN co-treatment.

TABLE 6
Numbers of cumulus oocyte complexes released from mice
treated with NMN alone, or cisplatin with or without NMN.

| | | | cisplatin + |
|---|---|---|---|
| ctrl | NMN | cisplatin | NMN |
| 21 | 13 | 10 | 21 |
| 13 | 14 | 4 | 14 |
| 16 | 11 | 9 | 16 |
| 23 | 16 | 10 | 15 |
| 16 | 14 | 5 | 11 |
| 19 | 17 | 6 | 18 |
| 20 | 16 | 0 | 19 |
| 14 | 12 | 10 | 16 |

Again, as in Example 2 above, the oocytes which were harvested from these studies were allowed to progress down meiosis, in order to assess meiotic progression rates (FIGS. 16 and 17 and Table 7 and 8).

TABLE 7
Proportions (numbers showing % of total) of harvested
oocytes achieving germinal vesicle breakdown at indicated time-
points, following release from IBMX.

| | | | | Cisplatin + |
|---|---|---|---|---|
| | Saline | NMN | cisplatin | NMN |
| 1 h | 30 | 45 | 43 | 53 |
| 2 h | 86 | 83 | 100 | 75 |

TABLE 8
Proportions (numbers showing % of total) of harvested
oocytes achieving polar body extrusion at indicated time -points,
following completion of GVBD.

| | | | | Cisplatin + |
|---|---|---|---|---|
| | Saline | NMN | cisplatin | NMN |
| 14 hr | 57 | 48 | 40 | 59 |
| 16 hr | 74 | 60 | 70 | 80 |
| 20 hr | 91 | 91 | 85 | 96 |

As above, there was no statistically significant difference in progression through either MI (as indicated by germinal vesicle breakdown, GVBD) or MII (as indicated by polar body extrusion). This indicates that the oocytes that do survive cisplatin treatment are meiotically competent, and that oocyte damage results in a binary “live/die” outcome.

### Example 4

### protection Against Doxorubicin Induced Infertility Through NMNAT1 and NMNAT3 Over-Expression

Following Example 2, where treatment with NMN was shown to protect against doxorubicin induced infertility, it was decided to further validate these results using mice which are genetically engineered to over-express the NAD⁺ biosynthetic enzymes NMNAT1 (which localizes to the nucleus) or NMNAT3 (which localizes to the mitochondria).

As in Example 2, 7 week-old female wild-type (“WT”) control or their littermates overexpressing NMNAT1 (“NMNAT1-Tg”) were treated with doxorubicin (10 mg/kg, i.p. injection). Unlike in Example 2, animals did not receive treatment with another compound, such as NMN. Two months later, animals were super-ovulated with PMSG, euthanased, and ovaries punctured to release MI oocytes, which were counted (FIG. 18 and Table 9).

TABLE 9
Oocyte yield following doxorubicin treatment (10 mg/kg,
i.p.) in wild-type mice, or mice genetically engineered to over-
express the nuclear NAD+ biosynthetic enzyme NMNAT1.

| | | ctrl- | Dox- |
|---|---|---|---|
| ctrl- | Dox- | NMNAT1- | NMNAT1- |
| WT | WT | Tg | Tg |
| 20 | 9 | 28 | 18 |
| 11 | 1 | 22 | 13 |
| 11 | 4 | 22 | 12 |
| 12 | 2 | 13 | 13 |
| 19 | 7 | 17 | 11 |
| 20 | 9 | 16 | 15 |
| 11 | 1 | 5 | 10 |
| 19 | 3 | 11 | 10 |
| 16 | 4 | 11 | |

Next, the same experiment above was repeated in mice which over-express the NAD+ biosynthetic gene NMNAT3, which is localized to the mitochondria (FIG. 19).

The data from this experiment shows that increasing the expression of two key NAD+ biosynthetic enzymes, NMNAT1 and NMNAT3, results in protection against doxorubicin induced infertility. These results match the results of experiments using pharmacological treatment with the cell permeable NAD precursor NMN, showing that increasing NAD through three different approaches results in protection against protect against chemotherapy induced infertility.

### Example 5

### Reversal of Chemotherapy Induced Infertility

In Examples 2-4, it was shown that co-treatment with NMN during chemotherapy treatment could protect against infertility. Next, we decided to investigate whether NMN treatment delivered some time following chemotherapy treatment could actively reverse chemotherapy induced infertility. As illustrated in FIG. 20, 8 week old C57BL6 females received chemotherapy (either doxorubicin, 10 mg/kg i.p., or cisplatin, 5 mg/kg i.p., or cyclophosphamide, 75 mg/kg i.p.) or a vehicle control at day 0. Four weeks later, they received NMN in their drinking water, for another 4 weeks, prior to being euthanased for either oocyte studies or ovarian histological analysis. The rationale of this experimental design is to investigate whether NMN has the ability to induce regeneration of the ovary. According to the standard dogma of reproductive biology, mammals are born with a set number of follicles which are slowly released as oocytes over the course of a lifetime, with the ovaries incapable of regenerating new follicles.

As shown in FIGS. 21 and 22 and Tables 10 and 11, NMN treatment delivered after chemotherapy results in a restoration of oocyte number.

TABLE 10
Primordial follicle numbers in ovarian histology
sections taken from mice treated with doxorubicin alone,
followed by NMN four weeks later.

| ctrl | NMN | Dox | Dox + NMN |
|---|---|---|---|
| 78 | 86 | 50 | 84 |
| 84 | 94 | 49 | 66 |
| 82 | 78 | 41 | 61 |
| 100 | 108 | 34 | 66 |
| 91 | | | |

TABLE 11
Oocyte yield in mice treated treated with cisplatin
alone, followed by NMN 4 weeks later, as described above and in
Supp Data 14. **p <0.01, 2 way ANOVA with Tukey test.

| | | | Cisptaitin + N |
|---|---|---|---|
| control | NMN | cisplatin | MN |
| 12 | 10 | 4 | 10 |
| 12 | 10 | 5 | 6 |
| 8 | 9 | 6 | 8 |
| 13 | 9 | 5 | 9 |
| | 17 | 2 | 7 |

As the ultimate functional measure of the ability to restore fertility, we measured the ability of mice to become pregnant when NMN was delivered substantially after chemotherapy (with cisplatin or cyclophosphamide—FIG. 23, FIG. 24 and Table 12), and observed a complete restoration in the number of pups born per litter when NMN was delivered well after the chemotherapy treatment period.

TABLE 12
Number of pups born per litter in mice treated with or
without cyclophosphamide (75 mg/kg, i.p. injection) at seven
weeks of age, followed four weeks later by treatment with the
NAD+ raising compound NMN for two months.

| | | | Cyclophos- |
|---|---|---|---|
| | | Cyclophos- | phamide |
| ctrl | NMN | phamide | NMN |
| 8 | 8 | 9 | 9 |
| 10 | 6 | 6 | 4 |
| 6 | 7 | 7 | 6 |
| 8 | 6 | 5 | 8 |
| 5 | 5 | 2 | 7 |
| 5 | | 1 | 7 |
| 8 | | 1 | 8 |
| 4 | | 1 | 7 |
| 6 | | 6 | 8 |
| 8 | | 1 | |
| 1 | | | |
| 6 | | | |
| 3 | | | |
| 4 | | | |

These data indicate the ability of NAD⁺ raising compounds to reverse, rather than just prevent, infertility caused by chemotherapy treatment.

These data support the hypothesis that ovaries are capable of regenerating new oocytes during adulthood, possibly through the existence of an oogonial stem cell. These data show that increasing NAD+ availability may represent the first know method for activating the differentiation of oogonial stem cells.

### Example 6

### preservation of Fertility with SIRT2 Over-Expression

As described above, SIRT2 is an NAD+ dependent deacylase enzyme which we have previously shown to deacetylate the kinetochore attachment protein BubRl. Levels of BubR1 decline with age in human oocytes, and this protein is rate limiting for the attachment of spindles to chromosomes, via their kinetochores. Decreased BubR1 levels and poor spindle attachment can impair meiosis, and mean an increased rate of chromosome mis-segregation, with oocytes suffering from too many or too few chromosomes (aneuploidy). We hypothesized that mice genetically engineered to over-express SIRT2 would have increased BubR1 levels in oocytes, which as a consequence, would maintain improved kinetochore attachment and function into old age. To generate a proof of concept for this principle, we generated a genetically engineered strain of mice which would constitutively over-express SIRT2 in all tissues (SIRT2-Tg). We then obtained oocytes from these animals, and performed a western blot to determine whether increased SIRT2 levels would alter BubR1 levels (FIG. 25). Consistent with our hypothesis, SIRT2 over-expression elevated BubR1 levels in oocytes.

Next, we obtained SIRT2-Tg female mice which were 14 months old. This is beyond the normal age of infertility for mice, which are fertile from 4 weeks of age, are discontinued from breeding from 7 months of age due to decreasing fertility, and are functionally infertile from 12 months of age, and by 15 months of age, have undergone complete ovarian failure (menopause). We first tested oocyte yield in mice from this age, and discovered that SIRT2-Tg mice had twice as many oocytes (COCs) as their WT littermates (FIG. 26 and Table 13).

TABLE 13
Oocyte (COC) yield in ovaries from 14 month-old WT
control or SIRT2-Tg mice.

| WT | SIRT2-Tg |
|---|---|
| 1.50 | 3.75 |
| 2.17 | 2.89 |
| 1.17 | 2.83 |
| 1.33 | 1.83 |
| 0.83 | 2.00 |
| 0.75 | 1.75 |
| 1.25 | 2.00 |
| 2.25 | 2.25 |
| 1.50 | 4.25 |

After determining that SIRT2-Tg maintained increased oocyte production into old age, we next sought to determine whether these oocytes were also of improved quality. To address this, we assessed the rates at which oocytes progressed through meiosis, using germinal vesicle breakdown (GVBD) rates as a marker for Meiosis I (FIG. 27, Table 14), and polar body extrusion (PBE) rates (FIG. 28, Table 15) as a marker for Meiosis II.

TABLE 14
Meiosis I progression rates, as determined by proportion
of oocytes achieving germinal vesicle breakdown,
in COCs from 14 month old WT control or SIRT2-Tg
mice. Numbers given are % of total oocytes.

| | WT | | | | | SIRT2-Tg | | | | |
|---|---|---|---|---|---|---|---|---|---|---|
| 0 | 0 | 0 | 0 | 0 | 0 | 0 | 0 | 0 | 0 | 0 |
| 1 | 33 | 0 | 29 | 0 | 0 | 93 | 8 | 6 | 0 | 63 |
| 1.5 | 15 | 71 | 25 | 50 | | 69 | 59 | 45 | 100 | |
| 2 | 67 | 62 | 86 | 38 | 100 | 93 | 92 | 88 | 64 | 100 |
| 3 | 67 | 85 | 86 | | | 93 | 100 | 88 | | |

TABLE 15
Meiosis II progression rates, as determined by proportion of oocytes
achieving polar body extrusion, in COCs from 14 month old WT control
or SIRT2-Tg mice. Numbers given are % of total oocytes.

| | WT | | | | SIRT2-Tg | | | |
|---|---|---|---|---|---|---|---|---|
| 10-12 hr | 11 | 0 | 54 | | 36 | 20 | 31 | |
| 12-14 hr | 27.3 | 11 | 0 | 61.5 | 57.6 | 57.1 | 46.7 | 61.5 |
| 14-16 hr | 50 | 27.3 | 11.1 | 14.3 | 63.2 | 66.7 | 64.3 | 60 |

SIRT2 overexpression resulted in a slightly increased rate of progression through GVBD, but a greatly improved rate of progression through PBE. Only 25% of oocytes from WT control animals progressed through PBE to complete meiotic maturation, versus 60% of SIRT2-Tg oocytes. Together, these data suggest that SIRT2 overexpression drastically improves oocyte meiotic competence during old age. These findings have important implications for the clinical treatment of infertility, whereby quality and ability of oocytes to progress through meiosis is essential to IVF success rates.

After determining that SIRT2 overexpression enhances oocyte yield, and showing that oocytes from aged SIRT2-Tg animals have an increased ability to complete meiosis, we next sought to determine whether SIRT2 overexpression could improve other parameters of health during old age. To address this, MII oocytes from aged animals were fixed and immunostained to highlight spindles, kinetochores, and chromosomes (FIG. 29). It was observed that spindle arrangement in oocytes from aged control (WT) animals demonstrates a chaotic structure, and it is highly unlikely that these oocytes will lead to a viable embryo, should fertilization occur. In contrast, oocytes from aged SIRT2-Tg littermates exhibit a classic bipolar, barrel shaped chromosome and spindle arrangement. These data are evidence of a drastic improvement in oocyte quality.

Poor spindle attachment as demonstrated in FIG. 29, due to decreased BubR1 levels, causes an increased risk of inaccurate chromosome segregation, with oocytes that have too many or too few chromosomes (aneuploidy). To assess the effects of SIRT2 overexpression on aneuploidy, we developed a monastrol-based protocol for counting chromosomes (FIG. 30). Consistent with our hypothesis, there was an increased incidence of aneuploidy between young and old animals, however overexpression of SIRT2 lowered the incidence of aneuploidy to that of young animals.

Another challenge faced by oocytes during aging is the ability to detoxify reactive oxygen species (ROS). SIRT2 is known to deacetylate and increase the activity of glucose 6 phosphate dehydrogenase (G6PD), an enzyme which regenerates glutathione, and detoxifies ROS. We next assessed the ability of oocytes to detoxify ROS by treating control (WT) and SIRT2-Tg oocytes with H₂O₂, and determining ROS levels using the stain DCFDA (FIG. 31). Strikingly, SIRT2 overexpression more than halved ROS levels, providing further evidence that this enzyme improves the health and resilience of oocytes. To directly assess whether this improved resilience against ROS was due to changes in G6PD activity, we then measured G6PD enzymatic activity. Assay conditions were as follows:
8 or 10 oocytes were used per assay;
Oocytes were lysed via freeze-thaw with liquid nitrogen;
7.5 mM G6P;
1.5 mM NADP;
buffer=Tris-HCl; pH7.4;
assay for G6PD activity was carried out at 25° C.;

The results are shown in FIG. 32. G6PD was indeed increased in oocytes from SIRT2-Tg mice, providing a mechanism for the ability of SIRT2 to improve resilience against ROS. To ultimately determine whether the preservation of oocyte quality during old age with SIRT2 overexpression would translate into improved fertility, we next performed mating trials in SIRT2-Tg and WT littermate control mice, which were aged to 16 months of age, well beyond the normal limits of fertility for mice. The design for this trial is as demonstrated in FIG. 12. Remarkably, the rate of fertility tripled, with 75% of aged SIRT2-Tg mice achieving pregnancies, compared to only 25% of their littermate controls (FIG. 33).

### Example 7

### Pharmacological Strategies to Mimic SIRT2 Overexpression and Improve Oocyte Quality

It was shown herein that genetic over-expression of the deacetylase SIRT2 resulted in protection against age induced infertility. As with other sirtuins, SIRT2 is critically dependent for its activity on levels of its enzymatic co-factor NAD⁺, which naturally declines with old age. We hypothesized that increasing NAD⁺ availability during old age could restore SIRT2 activity, and recapitulate the improvements in fertility observed with old age during SIRT2 over-expression. To test this, non-genetically modified C57BL6 female mice were treated with the NAD⁺ precursor nicotinamide mononucleotide (NMN), as in the Examples above. Mice were treated at 15 months of age, when mice normally experience ovarian failure (FIG. 34). Three weeks later, oocytes were harvested, and analysed for spindle structure (FIG. 35). Remarkably, only 3 weeks of NMN treatment was sufficient to reverse severe spindle defects, present in oocytes from untreated aged littermates. Moreover, oocyte yield was dramatically increased in mice treated with NMN as compared to control mice (FIG. 36). These data show that the benefits of SIRT2 over-expression can be mimicked through drug treatment.

### Example 8

### Treatment with an NAD Agonist Prior to IVF Improves Embryo Formation and Developmental Success

In addition to improving spindle quality and lowering aneuploidy rates, treating aged animals that have a background of impaired fertility leads to oocytes that have a better capacity for embryo development, as assessed by cell counts in blastocysts following in vitro fertilization (IVF). IVF is a common and clinically relevant procedure for couples who are unable to achieve unassisted pregnancy. Inner cell mass in embryos is an important predictor of subsequent implantation success rates.

In addition, the length of treatment with NAD raising compounds (e.g. NMN) positively correlates with improved fertility. Eight month old ex-breeder C57BL6 female mice, which are at an age of declining natural fertility, were treated with NMN through addition to drinking water (2 g/L) for 48 hours, 1 week, 2 weeks, and 4 weeks (6 per group). Animals were then treated with PMSG and then hCG to promote oocyte maturation and ovulation. MII stage oocytes were collected from the oviduct, and fertilized in vitro. Oocytes were then cultured into blastocysts for the next 6 days, at which point these blastocysts were subjected to differential staining to assess blastocyst cell number in either the inner cell mass or the trophectoderm. Results of counting inner cell mass cell numbers of blastocysts over 4 weeks are shown in FIG. 37. Inner cell mass cell number is accepted as an important predictor of implantation success rates.

Prolonged NMN exposure (addition to drinking water) leads to better efficacy, compared to single daily oral gavage. This indicates that the efficacy observed with NMN is an AUC (area under the curve) rather than a Cₘₐₓ pharmacokinetic effect. This was measured through performing IVF in oocytes obtained from 8 month old C57BL6 ex-breeder female mice, which were assigned to the following 4 groups (6 per group) . . .
Daily oral gavage, saline control, normal drinking water
Daily oral gavage, 10 mg NMN in saline, normal drinking water
No gavage, normal (control) drinking water
No gavage, NMN in drinking water (2 g/L)

These animals were at an age at which fertility normally declines in this species. Animals were maintained on each of the above treatments for 10 days. Animals were then treated with PMSG and then hCG to promote oocyte maturation and ovulation. MII stage oocytes were collected from the oviduct, and fertilized in vitro. Oocytes were then cultured into blastocysts for the next 6 days, and blastocyst formation rates were assessed. Results at days 5 and six are shown in FIGS. 38 and 39, respectively. Blastocyst formation and hatching were assessed as indicators of developmental competence. Blastocyst formation is a clinically relevant outcome: during clinical IVF, blastocyst stage and hatching blastocyst stage embryos are preferentially transferred over earlier stage embryos into a woman wishing to obtain a pregnancy.

1-63. (canceled)

64. A method of treating or preventing infertility in a female subject suffering from infertility or a decline in fertility, or at risk of suffering from infertility or a decline in fertility the method comprising administering to the subject an effective amount of an agent which elevates SIRT2 activity or SIRT2 expression in the subject.

65. The method of claim 64, wherein the treating or preventing comprises increasing fertility, or reducing rate of decline in fertility, or restoring fertility, of the female subject.

66. The method of claim 64, which comprises increasing oocyte quality, or reducing rate of decline in oocyte quality, in the female subject, and/or reducing the occurrence of aneuploidy in an oocyte of a female subject, and/or reducing the rate of decline in BubR1 activity in oocytes of a female subject suffering from a decline in fertility, or at risk of suffering from a decline in fertility, and/or promoting regeneration of ovarian follicles in an adult female subject.

67. The method of claim 64, which comprises an increase in pregnancy success rate of a female subject suffering from a decline in fertility.

68. The method of claim 64, wherein the agent which elevates SIRT2 activity or SIRT2 expression is an NAD⁺ agonist.

69. The method of claim 64, wherein the agent which elevates SIRT2 activity or SIRT2 expression is an NAD⁺ precursor.

70. The method of claim 69, wherein the NAD⁺ precursor is NMN or a pharmaceutically acceptable salt thereof, NR or a pharmaceutically acceptable salt thereof, NaR or a pharmaceutically acceptable salt thereof, NAAD or a pharmaceutically acceptable salt thereof, or NaMN or a pharmaceutically acceptable salt thereof.

71. The method of claim 68, wherein the NAD⁺ agonist is administered orally.

72. The method of claim 69, wherein the NAD⁺ precursor is administered orally.

73. The method of claim 64, wherein the subject is an aged subject.

74. The method of claim 1, wherein the subject is a subject who has been, is being, or will be, treated with chemotherapy, or has an underlying predisposition to infertility.

75. A method of increasing BubR1 activity in an oocyte, and/or increasing the fertilisation potential of an oocyte, comprising introducing into the oocyte an effective amount of an agent which elevates SIRT2 activity or SIRT2 expression in the oocyte.

76. A method according to claim 75, wherein the oocyte is in a female subject, and the agent which elevates SIRT2 activity or SIRT2 expression in the oocyte is introduced into the oocyte by administering the agent to the female subject.

77. The method of claim 75, wherein the agent which elevates SIRT2 activity or SIRT2 expression comprises an NAD⁺ precursor.

78. The method of claim 77, wherein the NAD⁺ precursor is NMN or a pharmaceutically acceptable salt thereof, NR or a pharmaceutically acceptable salt thereof, NaR or a pharmaceutically acceptable salt thereof, NAAD or a pharmaceutically acceptable salt thereof, or NaMN or a pharmaceutically acceptable salt thereof.

79. The method according to claim 75, wherein the method is carried out in vitro.

80. A method of fertilizing an oocyte in vitro, comprising introducing into the oocyte a donor sperm and an effective amount of an agent which elevates SIRT2 activity or SIRT2 expression in the oocyte.

81. The method of claim 80, wherein the agent which elevates SIRT2 activity or SIRT2 expression comprises an NAD⁺ precursor.

82. The method of claim 81, wherein the NAD⁺ precursor is NMN or a pharmaceutically acceptable salt thereof, NR or a pharmaceutically acceptable salt thereof, NaR or a pharmaceutically acceptable salt thereof, NAAD or a pharmaceutically acceptable salt thereof, or NaMN or a pharmaceutically acceptable salt thereof.

83. A method of increasing the probability that a zygote produced by fertilization of an oocyte in vitro will progress to a full term pregnancy following implantation, comprising introducing into the oocyte prior to, during, or after, fertilisation of the oocyte, an effective amount of an agent which elevates SIRT2 activity or SIRT2 expression in the oocyte.

84. The method of claim 83, wherein the agent which elevates SIRT2 activity or SIRT2 expression comprises an NAD⁺ precursor.

85. The method of claim 84, wherein the NAD⁺ precursor is NMN or a pharmaceutically acceptable salt thereof, NR or a pharmaceutically acceptable salt thereof, NaR or a pharmaceutically acceptable salt thereof, NAAD or a pharmaceutically acceptable salt thereof, or NaMN or a pharmaceutically acceptable salt thereof.

86. A method of improving or enhancing the ability of an oocyte to form a blastocyst during IVF, comprising introducing into the oocyte an effective amount of an agent which elevates SIRT2 activity or SIRT2 expression in a subject.

87. The method of claim 86, wherein the agent is an NAD+ precursor.

88. The method of claim 87, wherein the NAD⁺ precursor is NMN or a pharmaceutically acceptable salt thereof, NR or a pharmaceutically acceptable salt thereof, NaR or a pharmaceutically acceptable salt thereof, NAAD or a pharmaceutically acceptable salt thereof, or NaMN or a pharmaceutically acceptable salt thereof.

89. The method of claim 86, wherein the oocyte is in a female subject, and the agent is introduced into the oocyte by administering the agent to the female subject.

90. The method of claim 86, wherein the agent is introduced into the oocyte in vitro.


SEQUENCE LISTING (16 sequences)

SEQ ID NO: 1 - DNA, 1059 bp, Human
atggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtga

SEQ ID NO: 2 - protein, 389 aa, mouse
MAEPDPSDPLETQAGKVQEAQDSDSDTEGGATGGEAEMDFLRNLFTQTLGLGSQKERLLDELTLEGVTRYMQSERCRKVICLVGAGISTSAGIPDFRSPSTGLYANLEKYHLPYPEAIFEISYFKKHPEPFFALAKELYPGQFKPTICHYFIRLLKEKGLLLRCYTQNIDTLERVAGLEPQDLVEAHGTFYTSHCVNTSCRKEYTMGWMKEKIFSEATPRCEQCQSVVKPDIVFFGENLPSRFFSCMQSDFSKVDLLIIMGTSLQVQPFASLISKAPLATPRLLINKEKTGQTDPFLGMMMGLGGGMDFDSKKAYRDVAWLGDCDQGCLALADLLGWKKELEDLVRREHANIDAQSGSQAPNPSTTISPGKSPPPAKEAARTKEKEEQQ

SEQ ID NO: 3 - protein, 352 aa, human
MDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 4 - protein, 350 aa, rat
MDFLRNLFTQTLGLGSQKERLLDELTLEGVTRYMQSERCRRVICLVGAGISTSAGIPDFRSPSTGLYANLEKYHLPYPEAIFEISYFKKHPEPFFALAKELYPGQFKPTICHYFIRLLKEKGLLLRCYTQNIDTLERVAGLEPQDLVEAHGTFYTSHCVNTSCGKEYTMSWMKEKIFSEATPKCEKCQNVVKPDIVFFGENLPPRFFSCMQSDFSKVDLLIIMGTSLQVQPFASLISKAPLATPRLLINKEKTGQTDPFLGMMMGLGGGMDFDSKKAYRDVAWLGDCDQGCLALADLLGWKELEDLVRREHANIDAQSGSQASNPSATVSPRKSPPPAKEAARTKEKEEH

SEQ ID NO: 5 - protein, 491 aa, human NAMPT [sequence omitted]

SEQ ID NO: 6 - protein, 491 aa, mouse
MNAAAEAEFNILLATDSYKVTHYKQYPPNTSKVYSYFECREKKTENSKVRKVKYEETVFYGLQYILNKYLKGKVVTKEKIQEAKEVYREHFQDDVFNERGWNYILEKYDGHLPIEVKAVPEGSVIPRGNVLFTVENTDPECYWLTNWIETILVQSWYPITVATNSREQKKILAKYLLETSGNLDGLEYKLHDFGYRGVSSQETAGIGASAHLVNFKGTDTVAGIALIKKYYGTKDPVPGYSVPAAEHSTITAWGKDHEKDAFEHIVTQFSSVPVSVVSDSYDIYNACEKIWGEDLRHLIVSRSTEAPLIIRPDSGNPLDTVLKVLDILGKKFPVTENSKGYKLLPPYLRVIQGDGVDINTLQEIVEGMKQKKWSIENVSFGSGGALLQKLTRDLLNCSFKCSYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 7 - protein, 529 aa, Dog
MTDSGCPAPRQPSARGRRPRRAPGCGGARSWRDPSGPRSAPARAAASPGPAGSSSAQVTHYKQYPPNTSKVYSYFECREKKTENSKIKKVKYEETVFYGLQYILNKYLKGKVVTAEKIQEAKEVYREHFQDDVFNEKGWNYILEKYDGHLPIEIKAVPEGYVIPRGNVLFTVENTDPECYWLTNWIETILVQSWYPITVATNSREQKKILAKYLLETSGNLDGLEYKLHDFGYRGVSSQETAGIGASAHLVNFKGTDTVAGIAFVKKYYGTKDPVPGYSVPAAEHSTITAWGKDREKDAFEHIVTQFSSVPVSVVSDSYDIYNACEKIWGEDLRHLILSRTTEAPLIIRPDSGNPLDTVLKVLDILGKKFPITENSKGYKLLPPYLRVIQGDGVDINTLQEIVEGMKQKKWSIENIAFGSGGALLQKLTRDLLNCSFKCSYVVTNGLGINVFKDPVADPNKRSKKGRLSLHRTPAGNFVTLEEGKGDLEEYGHDLLHTVFKNGKVTKSYSFDEIRKNAKLNIELEVAPH

SEQ ID NO: 8 - protein, 279 aa, Human
MENSEKTEVVLLACGSFNPITNMHLRLFELAKDYMNGTGRYTVVKGIISPVGDAYKKKGLIPAYHRVIMAELATKNSKWVEVDTWESLQKEWKETLKVLRHHQEKLEASDCDHQQNSPTLERPGRKRKWTETQDSSQKKSLEPKTKAVPKVKLLCGADLLESFAVPNLWKSEDITQIVANYGLICVTRAGNDAQKFIYESDVLWKHRSNIHVVNEWIANDISSTKIRRALRRGQSIRYLVPDLVQEYIEKHNLYSSESEDRNAGVILAPLQRNTAEAKT

SEQ ID NO: 9 - protein, 285 aa, mouse
MDSSKKTEVVLLACGSFNPITNMHLRLFELAKDYMHATGKYSVIKGIISPVGDAYKKKGLIPAHHRIIMAELATKNSHWVEVDTWESLQKEWVETVKVLRYHQEKLATGSCSYPQSSPALEKPGRKRKWADQKQDSSPQKPQEPKPTGVPKVKLLCGADLLESFSVPNLWKMEDITQIVANFGLICITRAGSDAQKFIYESDVLWRHQSNIHLVNEWITNDISSTKIRRALRRGQSIRYLVPDLVQEYIEKHELYNTESEGRNAGVTLAPLQRNAAEAKHNHSTL

SEQ ID NO: 10 - protein, 279 aa, Dog
MENSKMEVVLLACGSFNPITNMHLRLFELAKDYMNGTGKYKVIKGIISPVGDAYKKKGLISAHHRVIMAELATKSSEWVEVDTWESLQKEWVETAKVLRHHQEKLEAGSCDHQQDSPVRGRPGQKRKWAEQRQDFSQKKSLEPKTKDVPKVKLLCGADLLESFGVPNLWKSEDITQIVGDYGLVCITRAGNDAQKFIYESDALWQHRNNIHLVNEWITNDISSTKIRRALRRGQSIRYLVPDLVQEYIEKHDLYSCESEERNVGVILAPLQRNTAEANS

SEQ ID NO: 11 - protein, 307 aa, Human
MTETTKTHVILLACGSFNPITKGHIQMFERARDYLHKTGRFIVIGGIVSPVHDSYGKQGLVSSRHRLIMCQLAVQNSDWIRVDPWECYQDTWQTTCSVLEHHRDLMKRVTGCILSNVNTPSMTPVIGQPQNETPQPIYQNSNVATKPTAAKILGKVGESLSRICCVRPPVERFTFVDENANLGTVMRYEEIELRILLLCGSDLLESFCIPGLWNEADMEVIVGDFGIVVVPRDAADTDRIMNHSSILRKYKNNIMVVKDDINHPMSVVSSTKSRLALQHGDGHVVDYLSQPVIDYILKSQLYINASG

SEQ ID NO: 12 - protein, 307 aa, mouse
MTETTKTHVILLACGSFNPITKGHIQMFERARDYLHKTGRFIVIGGIVSPVHDSYGKQGLVSSRHRLIMCQLAVQNSDWIRVDPWECYQDTWQTTCSVLEHHRDLMKRVTGCILSNVNTPSMTPVIGQPQHENTQPIYQNSNVPTKPTAAKILGKVGESLSRICCVRPPVERFTFVDENANLGTVMRYEEIELRILLLCGSDLLESFCIPGLWNEADMEVIVGDFGIVVVPRDAADTDRIMNHSSILRKYKNNIMVVKDDINHPMSVVSSTKSRLALQHGDGHVVDYLSQPVIDYILKSQLYINASG

SEQ ID NO: 13 - protein, 307 aa, Dog
MTETTKTHVILLACGSFNPITKGHIQMFERARDYLHKTGRFIVIGGIVSPVHDSYGKQGLVSSRHRLIMCQLAVQNSDWIRVDPWECYQDTWQTTCSVLEHHRDLMKRVTGCILSNVNTPSMTPVIGQPQNETPQPIYQNSNVSTKPTAAKILGKVGESLSRICCVRPPVERFTFVDENANLGTVMRYEEIELRILLLCGSDLLESFCIPGLWNEADMEVIVGDFGIVVVPRDAADTDRIMNHSSILRKYKNNIMVVKDDINHPMSVVSSTKSRLALQHGDGHVVDYLSQPVIDYILKSQLYINASG

SEQ ID NO: 14 - protein, 142 aa, Human
MEGPDHGKALFSTPAAVPELKLLCGADVLKTFQTPNLWKDAHIQEIVEKFGLVCVGRVGHDPKGYIAESPILRMHQHNIHLAKEPVQNEISATYIRRALGQGQSVKYLIPDAVITYIKDHGLYTKGSTWKGKSTQSTEGKTS

SEQ ID NO: 15 - protein, 179 aa, Mouse
MARLALQTSDWIRVDPWESEQAQWMETVKVLRHHHRELLRSSAQMDGPDPSKTPSASAALPELKLLCGADVLKTFQTPNLWKDTHIQEIVEKFGLVCVSRSGHDPERYISDSPILQQFQHNIHLAREPVLNEISATYVRKALGQGQSVKYLLPEAVITYIRDQGLYINDGSWKGKGKTG

SEQ ID NO: 16 - protein, 313 aa, Dog
MKSRIPVVLLACGSFNPITNMHLRLFEVARDHLHQTGWYFSLLSPVLVSRRQESLRSNLSRKQTSSLCQDGGIGLYQVIGGIISPVNDNYRKKDLVSAHHRVAMARLALQTSDWVRVDPWESEQVQWMETVKVLRTFLTQMSRKTVQHHHSELLRSLPQTEGLDHGRAGSTARTAGPELKLLCGADVLKTFQTPNLWKDAHIQEIVEKFGIVCVSRTGHNPKEYISGSPILHRYRHNIHLAREPVQNELSSTYVRQALSQGHSVKYLLPDAVIAYIKDHNLYTRDSSRKGSSTQRNEGKPSWGEPAPPPRSSS